(19) United States
(12) Patent Application Publication
(10) Pub. No.: US 2023/0307086 A1
(43) Pub. Date: Sep. 28, 2023

(54) METHODS AND SYSTEMS FOR DETERMINING DRUG EFFECTIVENESS

(71) Applicant: Algen Biotechnologies, Inc., San Francisco, CA (US)

(72) Inventors: Chun-Hao Huang, San Francisco, CA (US); Spencer Charles Knight, San Francisco, CA (US); Ko-Chuan Lee, San Francisco, CA (US)

(21) Appl. No.: 18/099,526

(22) Filed: Jan. 20, 2023

Related U.S. Application Data
(63) Continuation of application No. PCT/US2021/042537, filed on Jul. 21, 2021.
(60) Provisional application No. 63/054,890, filed on Jul. 22, 2020.

(51) Int. Cl. G16B 5/00; G06F 30/27; G16B 20/30; G16B 15/30; G16B 40/20

(52) U.S. Cl. CPC G16B 5/00; G06F 30/27; G16B 15/30; G16B 20/30; G16B 40/20

(57) ABSTRACT

Methods and systems for determining an effectiveness of a drug (e.g., on- and off-target effects) may comprise: generating a latent space representation, which represents phenotypic states of a cell type, of nucleic acid sequence data for diseased and normal cells of the cell type; identifying, based at least in part on the latent space topology, a target genomic region; mapping sequence data of a first cell of the cell type, which has been modified, to the latent space to yield a first latent space representation; mapping sequence data of a second cell of the cell type, which has been exposed to the drug and exhibited the first phenotypic state before exposure, to the latent space to yield a second latent space representation; and determining, based at least in part on the first and second latent space representations, the effectiveness of the drug.

### CROSS-REFERENCE

This application is a continuation of International Application No. PCT/US2021/042537, filed Jul. 21, 2021, which claims the benefit of U.S. Provisional Application No. 63/054,890, filed Jul. 22, 2020, each of which is incorporated by reference herein in its entirety.

### BACKGROUND

The ability to evaluate the on- and off-targets of a drug may hold great promise for therapeutic applications. However, this may be a challenging task and may require extensive, time-intensive experimental assays and animal models for each target gene of interest. Further, therapeutic targeting using drugs, such as treatment inhibitors, may be evaluated for effectiveness in subjects with a disease or disorder.

### SUMMARY

Recognized herein is a need for improved methods for evaluating on- and off-targets of a drug, which may affect its effectiveness. Such drugs may be associated with certain genomic regions that are suitable for therapeutic targeting. Methods and systems provided herein may significantly increase the efficiency, accuracy, and/or throughput of determining the on- and off-targets of drugs. Such methods and systems may leverage the identification of certain genomic regions for therapeutic targeting.

The present disclosure provides methods and systems for evaluating on- and off-targets of a drug. Such drugs may be associated with target genomic regions. For example, the present technology relates to high-throughput screening of drug candidates, which may leverage high-content, high-efficiency, and high-throughput CRISPR (clustered regularly interspaced short palindromic repeats) screening techniques for identifying relevant target genes that may be selected as effective therapeutic targets. These screens may leverage suitable algorithms to compare single-cell transcriptomic fingerprints of drugs for each gene that is targeted via CRISPR. Methods and systems of the present disclosure may rapidly and accurately evaluate on- and off-targets of a drug, based at least in part on quantification of the ability to selectively modify target genomic regions of cells as a basis for choosing biomarkers and therapeutic targets relevant to a disease indication of interest. Such methods and systems may comprise selecting drugs which have a high therapeutic index by comparing their fingerprints with toxic fingerprints generated by CRISPR targeting essential genes (e.g., RPA1).

The ability to selectively modify target genomic regions of cells to alter their cellular states (e.g., by converting cells from one differentiated state to another) may hold great promise for therapeutic applications. However, despite the promise of selective modification of cellular states (e.g., via cellular re-programming), the identification of genetic drivers that may mediate the transition between one cell state to another remains challenging for many therapeutically relevant applications. For example, the phenotype of re-programming may be complex and may involve many genes interacting with each other in a hierarchical, non-linear fashion. Disentangling which of these genes is causal versus correlative in a given process may be a challenging task and may require extensive, time-intensive experimental assays and animal models for each gene of interest. Further, therapeutic targeting using drugs, such as treatment inhibitors, may be evaluated for effectiveness in subjects with a disease or disorder.

Also recognized herein is a need for improved methods for determining an effectiveness of a drug. Such drugs may be associated with certain genomic regions that are suitable for therapeutic targeting (e.g., genomic regions which may facilitate re-programming of a cell from one phenotypic state to another). Methods and systems provided herein may significantly increase the efficiency, accuracy, and/or throughput of determining the effectiveness of drugs. Such methods and systems may leverage the identification of certain genomic regions for therapeutic targeting.

The present disclosure further provides methods and systems for determining an effectiveness of a drug. Such drugs may be associated with target genomic regions of cells that may be selectively modified to alter their cellular states (e.g., via transcriptional re-programming of cells from one differentiated state to another). For example, the present technology relates to high-throughput screening of drug candidates, which may leverage high-content, high-efficiency, and high-throughput CRISPR (clustered regularly interspaced short palindromic repeats) screening techniques for identifying relevant target genes that may potentially mediate re-programming between phenotypically distinct cellular states and/or be selected as effective therapeutic targets. These screens may leverage anomaly detection models to quantify re-programming as a measurable phenotype for each gene that is targeted via CRISPR. Methods and systems of the present disclosure may effectively determine an effectiveness of a drug, based at least in part on quantification of the ability to selectively modify target genomic regions of cells (e.g., via cellular re-programming) as a basis for choosing biomarkers and therapeutic targets relevant to a disease indication of interest.

In an aspect, the present disclosure provides a method for determining an effectiveness of a drug, comprising: (a) generating a latent space representation of nucleic acid sequence data for a plurality of diseased cells and a plurality of normal cells of a cell type, wherein said latent space represents a plurality of phenotypic states of said cell type; (b) identifying, based at least in part on a topology of said latent space, a genomic region that facilitates reprogramming of said cell type from a first phenotypic state to a second phenotypic state of said plurality of phenotypic states; (c) mapping sequence data of a first cell of said cell type to said latent space to yield a first latent space representation, wherein said first cell has been reprogrammed from said first phenotypic state to said second phenotypic state; (d) mapping sequence data of a second cell of said cell type to said latent space to yield a second latent space representation, wherein said second cell has been exposed to said drug, and wherein prior to said second cell being exposed to said drug, said second cell exhibited said first phenotypic state; and (e) determining, based at least in part on said first latent space representation and said second latent space representation, said effectiveness of said drug.

In some embodiments, (a) comprises using a supervised dimensionality reduction algorithm to generate said latent space representation. In some embodiments, said supervised dimensionality reduction algorithm is a uniform manifold approximation and projection (UMAP) algorithm. In some embodiments, said supervised dimensionality reduction algorithm is a t-distributed stochastic neighbor embedding (t-SNE) algorithm. In some embodiments, said supervised dimensionality reduction algorithm is a variable autoencoder. In some embodiments, (b) comprises performing non-linear cell trajectory reconstruction on said latent space to construct an inferred maximum likelihood progression trajectory between said first phenotypic state and said second phenotypic state. In some embodiments, performing said non-linear cell trajectory reconstruction comprises applying a reverse graph embedding algorithm to said latent space.

In some embodiments, said first phenotypic state is cancer and said second phenotypic state is a wildtype state. In some embodiments, said second phenotypic state is an intermediate state. In some embodiments, said intermediate state is a fibroblast state or a progenitor cell state. In some embodiments, said first cell has been reprogrammed from said first phenotypic state to said second phenotypic state using genetic editing. In some embodiments, said genetic editing is performed with a genetic editing unit selected from the group consisting of a CRISPR (e.g., active Cas9) system, a CRISPRi (e.g., CRISPR interference, a catalytically dead Cas9 fused to a transcriptional repressor peptide including KRAB) system, a CRISPRa (e.g., CRISPR activation, a catalytically dead Cas9 fused to a transcriptional activator peptide including VPR (HIV viral protein R)) system, a RNAi system, and a shRNA system.

In some embodiments, (e) comprises measuring (i) a shift in said latent space representation of said first cell from said editing and (ii) a shift in said latent space representation of said second cell from said exposure to said drug; and mathematically relating (i) to (ii). In some embodiments, said measuring comprises using a supervised learning algorithm. In some embodiments, said supervised learning algorithm is a support vector machine, a random forest, logistic regression, a Bayesian classifier, or a convolutional neural network.

In some embodiments, the method further comprises: mapping nucleic acid sequence data of a plurality of additional cells of said cell type to said latent space, wherein each cell of said plurality of additional cells has been exposed to a respective drug of a plurality of drugs; determining, based at least in part on said latent space representation of said first cell and latent space representations of said plurality of additional cells, an effectiveness of each drug; and electronically outputting a ranking of said plurality of drugs based at least in part on said effectiveness of each drug. In some embodiments, said drug is selected from the group consisting of: a compound (e.g., a small molecule), an inhibitor (e.g., a small molecule inhibitor), and an antibody.

In some embodiments, at least one of said sequence data of said first cell of said cell type and said sequence data of said second cell of said cell type is generated by single-cell sequencing. In some embodiments, at least one of said sequence data of said first cell of said cell type and said sequence data of said second cell of said cell type is generated by sequential single-cell sequencing.

In another aspect, the present disclosure provides a method for determining an effectiveness of a drug, comprising: (a) generating a latent space representation of nucleic acid sequence data for a plurality of diseased cells and a plurality of normal cells of a cell type, wherein said latent space represents a plurality of phenotypic states of said cell type; (b) identifying, based at least in part on a topology of said latent space, a target genomic region of said cell type; (c) mapping sequence data of a first cell of said cell type to said latent space to yield a first latent space representation, wherein said target genomic region of said first cell has been modified, and wherein said first cell exhibited a first phenotypic state prior to said modification; (d) mapping sequence data of a second cell of said cell type to said latent space to yield a second latent space representation, wherein said second cell has been exposed to said drug, and wherein prior to said second cell being exposed to said drug, said second cell exhibited said first phenotypic state; and (e) determining, based at least in part on said first latent space representation and said second latent space representation, said effectiveness of said drug.

In some embodiments, (a) comprises using a supervised dimensionality reduction algorithm to generate said latent space representation. In some embodiments, said supervised dimensionality reduction algorithm is a uniform manifold approximation and projection (UMAP) algorithm. In some embodiments, said supervised dimensionality reduction algorithm is a t-distributed stochastic neighbor embedding (t-SNE) algorithm. In some embodiments, said supervised dimensionality reduction algorithm is a variable autoencoder.

In some embodiments, said first phenotypic state is cancer. In some embodiments, said first phenotypic state is an intermediate state. In some embodiments, said intermediate state is a fibroblast state or a progenitor cell state.

In some embodiments, (e) comprises measuring (i) a shift in said latent space representation of said first cell from said modification, and (ii) a shift in said latent space representation of said second cell from said exposure to said drug; and mathematically relating (i) to (ii). In some embodiments, said measuring comprises using a supervised learning algorithm. In some embodiments, said supervised learning algorithm is a support vector machine, a random forest, logistic regression, a Bayesian classifier, or a convolutional neural network.

In some embodiments, the method further comprises: mapping nucleic acid sequence data of a plurality of additional cells of said cell type to said latent space, wherein each cell of said plurality of additional cells has been exposed to a respective drug of a plurality of drugs; determining, based at least in part on said latent space representation of said first cell and latent space representations of said plurality of additional cells, an effectiveness of each drug; and electronically outputting a ranking of said plurality of drugs based at least in part on said effectiveness of each drug. In some embodiments, said drug is selected from the group consisting of: a compound (e.g., a small molecule), an inhibitor (e.g., a small molecule inhibitor), and an antibody.

In some embodiments, at least one of said sequence data of said first cell of said cell type and said sequence data of said second cell of said cell type is generated by single-cell sequencing. In some embodiments, at least one of said sequence data of said first cell of said cell type and said sequence data of said second cell of said cell type is generated by sequential single-cell sequencing.

In some embodiments, the modification in (c) comprises use of a genetic editing unit. In some embodiments, the genetic editing is performed with a genetic editing unit selected from the group consisting of a CRISPR system, a CRISPRi system, a CRISPRa system, a RNAi system, and a shRNA system. In some embodiments, the modification in (c) comprises use of a single-guide RNA (sgRNA) that targets at least a portion of the target genomic region. In some embodiments, (e) comprises comparing the first latent space representation to the second latent space representation. In some embodiments, (e) comprises determining the effectiveness of the drug based at least in part on determining a maximal similarity of the first latent space representation to an on-target latent space representation or a minimal similarity of the first latent space representation to an off-target latent space representation.

In another aspect, the present disclosure provides a system for determining an effectiveness of a drug, comprising: a database that comprises nucleic acid sequence data for a plurality of diseased cells and a plurality of normal cells of a cell type; and one or more computer processors that are individually or collectively programmed to: (i) generate a latent space representation of said nucleic acid sequence data, wherein said latent space represents a plurality of phenotypic states of said cell type; (ii) identify, based at least in part on a topology of said latent space, a genomic region that facilitates reprogramming of said cell type from a first phenotypic state to a second phenotypic state of said plurality of phenotypic states; (iii) map sequence data of a first cell of said cell type to said latent space to yield a first latent space representation, wherein said first cell has been reprogrammed from said first phenotypic state to said second phenotypic state; (iv) map sequence data of a second cell of said cell type to said latent space to yield a second latent space representation, wherein said second cell has been exposed to said drug, and wherein prior to said second cell being exposed to said drug, said second cell exhibited said first phenotypic state; and (v) determine, based at least in part on said first latent space representation and said second latent space representation, said effectiveness of said drug.

In another aspect, the present disclosure provides a non-transitory computer-readable medium comprising machine-executable code that, upon execution by one or more computer processors, implements a method for determining an effectiveness of a drug, said method comprising: (a) generating a latent space representation of nucleic acid sequence data for a plurality of diseased cells and a plurality of normal cells of a cell type, wherein said latent space represents a plurality of phenotypic states of said cell type; (b) identifying, based at least in part on a topology of said latent space, a genomic region that facilitates reprogramming of said cell type from a first phenotypic state to a second phenotypic state of said plurality of phenotypic states; (c) mapping sequence data of a first cell of said cell type to said latent space to yield a first latent space representation, wherein said first cell has been reprogrammed from said first phenotypic state to said second phenotypic state; (d) mapping sequence data of a second cell of said cell type to said latent space to yield a second latent space representation, wherein said second cell has been exposed to said drug, and wherein prior to said second cell being exposed to said drug, said second cell exhibited said first phenotypic state; and (e) determining, based at least in part on said first latent space representation and said second latent space representation, said effectiveness of said drug.

In another aspect, the present disclosure provides a system for determining an effectiveness of a drug, comprising: a database that comprises nucleic acid sequence data for a plurality of diseased cells and a plurality of normal cells of a cell type; and one or more computer processors that are individually or collectively programmed to: (i) generate a latent space representation of said nucleic acid sequence data, wherein said latent space represents a plurality of phenotypic states of said cell type; (ii) identify, based at least in part on a topology of said latent space, a target genomic region of said cell type; (iii) map sequence data of a first cell of said cell type to said latent space to yield a first latent space representation, wherein said target genomic region of said first cell has been modified, and wherein said first cell exhibited a first phenotypic state prior to said modification; (iv) map sequence data of a second cell of said cell type to said latent space to yield a second latent space representation, wherein said second cell has been exposed to said drug, and wherein prior to said second cell being exposed to said drug, said second cell exhibited said first phenotypic state; and (v) determine, based at least in part on said first latent space representation and said second latent space representation, said effectiveness of said drug.

In another aspect, the present disclosure provides a non-transitory computer-readable medium comprising machine-executable code that, upon execution by one or more computer processors, implements a method for determining an effectiveness of a drug, said method comprising: (a) generating a latent space representation of nucleic acid sequence data for a plurality of diseased cells and a plurality of normal cells of a cell type, wherein said latent space represents a plurality of phenotypic states of said cell type; (b) identifying, based at least in part on a topology of said latent space, a target genomic region of said cell type; (c) mapping sequence data of a first cell of said cell type to said latent space to yield a first latent space representation, wherein said target genomic region of said first cell has been modified, and wherein said first cell exhibited a first phenotypic state prior to said modification; (d) mapping sequence data of a second cell of said cell type to said latent space to yield a second latent space representation, wherein said second cell has been exposed to said drug, and wherein prior to said second cell being exposed to said drug, said second cell exhibited said first phenotypic state; and (e) determining, based at least in part on said first latent space representation and said second latent space representation, said effectiveness of said drug.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIGS. 1A-1B show examples of flowcharts illustrating methods for determining an effectiveness of a drug.

FIG. 2 shows a computer system that is programmed or otherwise configured to implement methods provided herein.

FIG. 3A shows an example of assessing drugs’ on- and off-target effects and identification of novel inhibitors. By leveraging CRISPRi gene interrogation, sequential single-cell sequencing, intelligent latent space construction, and supervised learning, on- and off-target effects from drug fingerprints (inhibition of targets by small molecules, antibodies) are assessed in accordance with their ability to match a desired state dictated by target fingerprints (interrogation of targets by CRISPRi, CRISPR, RNAi).

FIG. 3B shows an illustration of supervised learning as a method for training model on binary cell types to classify new cells by comparing classifications with original and desired states.

FIGS. 4A-4B show an example of a sequential single-cell sequencing approach to normalize reads and gene numbers across samples, including a schematic illustration of the normalization approach (FIG. 4A), and a number of reads and genes per cell from samples before and after the sequential single-cell sequencing approach (FIG. 4B); DMSO indicates that MIAPaCa-2 cells were treated with DMSO for 6 hours; Piper indicates that MIAPaCa-2 cells were treated with Piperlongumine for 6 hours.

FIGS. 5A-5D show an example of machine-learning-driven selection of top drug candidates based on quantification of single-cell RNA-sequencing profiles (6-hour treatment). FIGS. 5A-5B show 2-dimensional UMAP projections of the human cancer pancreatic cancer cells MIAPaCa-2 and healthy pancreatic duct cells hTERT-HPNE shown by either cell type (FIG. 5A) or drug treatment (Auranofin, D9, or Piperlongumine) and duration (FIG. 5B). FIG. 5C shows machine learning classification of cells treated with either vehicle controls (DMSO) or drug candidates. Briefly, supervised machine learning algorithms were trained on 2-dimensional UMAP transcriptome profiles of pure cell types (healthy and cancer cell) to allow for binary discrimination between cell types with an AUC exceeding 0.98. Treated cells were then assigned as “cancer” or “healthy” on the basis of their resulting 2-dimensional transcriptomes following treatment. FIG. 5D shows a summary of binomial testing results for drug candidates relative to a vehicle control (DMSO).

FIGS. 6A-6D show an example of machine-learning-driven selection of top drug candidates based on quantification of single-cell RNA-sequencing profiles (24-hour treatment). FIGS. 6A-6B show 2-dimensional UMAP projections of the human cancer pancreatic cancer cells MIAPaCa-2 and healthy pancreatic duct cells hTERT-HPNE shown by either cell type (FIG. 6A) or drug treatment (Auranofin, D9, or Piperlongumine) and duration (FIG. 6B). FIG. 6C shows machine learning classification of cells treated with either vehicle controls (DMSO) or drug candidates. Briefly, supervised machine learning algorithms were trained on 2-dimensional UMAP transcriptome profiles of pure cell types (healthy and cancer cell) to allow for binary discrimination between cell types with an AUC exceeding 0.98. Treated cells were then assigned as “cancer” or “healthy” on the basis of their resulting 2-dimensional transcriptomes following treatment. FIG. 6D shows a summary of binomial testing results for drug candidates relative to a vehicle control (DMSO).

FIG. 7 shows an illustration of supervised learning as a method for training model on binary cell types to classify new drug-treated cells by comparing classifications with cells which have on- and off-targets interrogated by CRISPR.

FIGS. 8A-8H show examples of assessing drugs’ on- and off-target effects. 2-dimensional UMAP projections of human pancreatic cancer cell lines MIAPaCa-2 (which may be shown to be dependent on KRAS and TXNRD1 signaling) were shown by sgRNA (including negative control sgRNA in FIG. 8A, KRAS sgRNA in FIG. 8B, TXNRD1 sgRNA in FIG. 8C, and RPA1 sgRNA in FIG. 8D) or drug treatments (including Auranofin in FIG. 8E, D9 in FIG. 8F, and Piperlongumine in FIG. 8G) or merged (FIG. 8H). As shown by the dash line circles in FIG. 8H, on- and off-target effects from pharmacological inhibition (TXNRD1 inhibited by Auranofin, D9, or Piperlongumine) were assessed in accordance with their ability to match an on-target fingerprint dictated by genetic inhibition (sgRNAs targeting TXNRD1 or KRAS). sgRNA targeting an essential gene RPA1 was used as a toxic control fingerprint.

FIGS. 9A-9H show examples of assessing drugs’ on- and off-target effects. 2-dimensional t-Distributed Stochastic Neighbor Embedding (t-SNE) projections of human pancreatic cancer cell lines MIAPaCa-2 (which may be shown to be dependent on KRAS and TXNRD1 signaling) were shown by sgRNA (including negative control sgRNA in FIG. 9A, KRAS sgRNA in FIG. 9B, TXNRD1 sgRNA in FIG. 9C, and RPA1 sgRNA in FIG. 9D) or drug treatments (including Auranofin in FIG. 9E, D9 in FIG. 9F, and Piperlongumine in FIG. 9G) or merged (FIG. 9H). As shown by the dash line circles in FIG. 9H, on- and off-target effects from pharmacological inhibition (TXNRD1 inhibited by Auranofin, D9, or Piperlongumine) were assessed in accordance with their ability to match an on-target fingerprint dictated by genetic inhibition (sgRNAs targeting TXNRD1 or KRAS). sgRNA targeting an essential gene RPA1 was used as a toxic control fingerprint.

FIGS. 10A-10F show the reproducibility of this method to assess drugs’ on- and off-target effects using TXNRD1 target gene as an example. 2-dimensional UMAP projections of human pancreatic cancer cell lines MIAPaCa-2 (which may be shown to be dependent on KRAS and TXNRD1 signaling) were shown by sgRNA (including negative control sgRNA in FIG. 10A, TXNRD1 #1 sgRNA in FIG. 10B, and TXNRD1 #2 sgRNA in FIG. 10C) or drug treatments (including Auranofin in FIG. 10D) or merged (FIG. 10E). As shown by the dash line circles in FIG. 10E, on- and off-target effects from pharmacological inhibition (TXNRD1 inhibited by Auranofin) were assessed in accordance with their ability to match on-target fingerprints dictated by two independent genetic inhibition (two independent sgRNAs targeting TXNRD1). Quantitative PCR (qPCR) analysis of TXNRD1 gene expression in human pancreatic cancer cell lines MIAPaCa-2 transduced with two independent sgRNAs targeting TXNRD1 is shown in FIG. 10F. Data are presented as mean ± standard deviation. Statistical significance between groups was calculated by two-tailed Student’s t-test. Significance value is P < 0.05 (*).

FIGS. 11A-11F show the reproducibility of this method to assess drugs’ on- and off-target effects using KRAS target gene as an example. 2-dimensional UMAP projections of human pancreatic cancer cell lines MIAPaCa-2 (which may be shown to be dependent on KRAS and TXNRD1 signaling) were shown by sgRNA (including negative control sgRNA in FIG. 11A, KRAS #1 sgRNA in FIG. 11B, and KRAS #2 sgRNA in FIG. 11C) or drug treatments (including Auranofin in FIG. 11D) or merged (FIG. 11E). As shown by the dash line circles in FIG. 11E, on-and off-target effects from pharmacological inhibition (Auranofin) were assessed in accordance with their ability to match on-target fingerprints dictated by two independent genetic inhibition (two independent sgRNAs targeting KRAS). Quantitative PCR (qPCR) analysis of KRAS gene expression in human pancreatic cancer cell lines MIAPaCa-2 transduced with two independent sgRNAs targeting KRAS is shown in FIG. 11F. Data are presented as mean ± standard deviation. Statistical significance between groups was calculated by two-tailed Student’s t-test. Significance values are P < 0.05 (*) and P < 0.01 (**).

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “sequencing,” as used herein, generally refers to a process for generating or identifying a sequence of a biological molecule, such as a nucleic acid molecule. Such sequence may be a nucleic acid sequence, which may include a sequence of nucleic acid bases. Sequencing methods may be massively parallel array sequencing (e.g., Illumina sequencing), which may be performed using template nucleic acid molecules immobilized on a support, such as a flow cell or beads. Sequencing methods may include, but are not limited to: high-throughput sequencing, next-generation sequencing, sequencing-by-synthesis, flow sequencing, massively-parallel sequencing, shotgun sequencing, single-molecule sequencing, nanopore sequencing, pyrosequencing, semiconductor sequencing, sequencing-by-ligation, sequencing-by-hybridization, RNA-Seq (Illumina), Digital Gene Expression (Helicos), Single Molecule Sequencing by Synthesis (SMSS) (Helicos), Clonal Single Molecule Array (Solexa), and Maxim-Gilbert sequencing.

The term “subject,” as used herein, generally refers to an individual having a biological sample that is undergoing processing or analysis. A subject may be an animal or plant. The subject may be a mammal, such as a human, ape, monkey, chimpanzee, dog, cat, horse, pig, rodent (e.g., mouse or rat), reptile, amphibian, or bird. The subject may have or be suspected of having a disease, such as cancer (e.g., breast cancer, colorectal cancer, brain cancer, leukemia, lung cancer, skin cancer, liver cancer, pancreatic cancer, lymphoma, esophageal cancer, or cervical cancer) or an infectious disease.

The term “sample,” as used herein, generally refers to a biological sample. Examples of biological samples include tissues, cells, nucleic acid molecules, amino acids, polypeptides, proteins, carbohydrates, fats, metabolites, hormones, and viruses. In an example, a biological sample is a nucleic acid sample including one or more nucleic acid molecules, such as deoxyribonucleic acid (DNA) and/or ribonucleic acid (RNA). The nucleic acid molecules may be cell-free or cell-free nucleic acid molecules, such as cell-free DNA or cell-free RNA. The nucleic acid molecules may be derived from a variety of sources including human, mammal, non-human mammal, ape, monkey, chimpanzee, reptilian, amphibian, or avian, sources. Further, samples may be extracted from variety of animal fluids containing cell-free sequences, including but not limited to blood, serum, plasma, vitreous, sputum, urine, tears, perspiration, saliva, semen, mucosal excretions, mucus, spinal fluid, amniotic fluid, lymph fluid and the like. Cell-free polynucleotides may be fetal in origin (via fluid taken from a pregnant subject), or may be derived from tissue of the subject itself.

The term “nucleic acid,” or “polynucleotide,” as used herein, generally refers to a molecule comprising one or more nucleic acid subunits, or nucleotides. A nucleic acid may include one or more nucleotides selected from adenosine (A), cytosine (C), guanine (G), thymine (T) and uracil (U), or variants thereof. A nucleotide generally includes a nucleoside and at least 1, 2, 3, 4, 5, 6, 7, 8, 9, 10, or more phosphate (PO₃) groups. A nucleotide may include a nucleobase, a five-carbon sugar (either ribose or deoxyribose), and one or more phosphate groups.

Ribonucleotides are nucleotides in which the sugar is ribose. Deoxyribonucleotides are nucleotides in which the sugar is deoxyribose. A nucleotide may be a nucleoside monophosphate or a nucleoside polyphosphate. A nucleotide may be a deoxyribonucleoside polyphosphate, such as, e.g., a deoxyribonucleoside triphosphate (dNTP), which may be selected from deoxyadenosine triphosphate (dATP), deoxycytidine triphosphate (dCTP), deoxyguanosine triphosphate (dGTP), uridine triphosphate (dUTP) and deoxythymidine triphosphate (dTTP) dNTPs, that include detectable tags, such as luminescent tags or markers (e.g., fluorophores). A nucleotide may include any subunit that may be incorporated into a growing nucleic acid strand. Such subunit may be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). In some examples, a nucleic acid is deoxyribonucleic acid (DNA), ribonucleic acid (RNA), or derivatives or variants thereof. A nucleic acid may be single-stranded or double-stranded. In some cases, a nucleic acid molecule is circular.

The terms “nucleic acid molecule,” “nucleic acid sequence,” “nucleic acid fragment,” “oligonucleotide” and “polynucleotide,” as used herein, generally refer to a polynucleotide that may have various lengths, such as either deoxyribonucleotides or ribonucleotides (RNA), or analogs thereof. A nucleic acid molecule may have a length of at least about 10 bases, 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. An oligonucleotide may be composed of a specific sequence of four nucleotide bases: adenine (A); cytosine (C); guanine (G); and thymine (T) (uracil (U) for thymine (T) when the polynucleotide is RNA). Thus, the term “oligonucleotide sequence” is the alphabetical representation of a polynucleotide molecule; alternatively, the term may be applied to the polynucleotide molecule itself. This alphabetical representation may be input into databases in a computer having a central processing unit and used for bio informatics applications such as functional genomics and homology searching. Oligonucleotides may include one or more nonstandard nucleotide(s), nucleotide analog(s), and/or modified nucleotides.

The term “nucleotide analogs,” as used herein, may include, but are not limited to, diaminopurine, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D- mannosylqueosine, 5′-methoxycarboxymethyluracil, 5-methoxyuracil, 2-methylthio-D46- isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5- oxyacetic acid methylester, uracil-5-oxyacetic acid(v), 5-methyl-2-thiouracil, 3-(3-amino- 3- N-2-carboxypropyl) uracil, (acp3)w, 2,6-diaminopurine, phosphoroselenoate nucleic acids, and the like. In some cases, nucleotides may include modifications in their phosphate moieties, including modifications to a triphosphate moiety. Additional, non-limiting examples of modifications include phosphate chains of greater length (e.g., a phosphate chain having 4, 5, 6, 7, 8, 9, 10, or more than 10 phosphate moieties), modifications with thiol moieties (e.g., alpha-thio triphosphate and beta-thiotriphosphates) or modifications with selenium moieties (e.g., phosphoroselenoate nucleic acids). Nucleic acid molecules may also be modified at the base moiety (e.g., at one or more atoms that may be available to form a hydrogen bond with a complementary nucleotide and/or at one or more atoms that may not be capable of forming a hydrogen bond with a complementary nucleotide), sugar moiety or phosphate backbone. Nucleic acid molecules may also contain amine-modified groups, such as aminoallyl-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure may provide higher density in bits per cubic millimeter (mm), higher safety (e.g., resistance to accidental or purposeful synthesis of natural toxins), easier discrimination in photo-programmed polymerases, or lower secondary structure. Nucleotide analogs may be capable of reacting or bonding with detectable moieties for nucleotide detection.

The term “free nucleotide analog” as used herein, generally refers to a nucleotide analog that is not coupled to an additional nucleotide or nucleotide analog. Free nucleotide analogs may be incorporated in to the growing nucleic acid chain by primer extension reactions.

The term “primer(s),” as used herein, generally refers to a polynucleotide which is complementary to the template nucleic acid. The complementarity or homology or sequence identity between the primer and the template nucleic acid may be limited. The length of the primer may be between 8 nucleotide bases to 50 nucleotide bases. The length of the primer may be greater than or equal to 6 nucleotide bases, 7 nucleotide bases, 8 nucleotide bases, 9 nucleotide bases, 10 nucleotide bases, 11 nucleotide bases, 12 nucleotide bases, 13 nucleotide bases, 14 nucleotide bases, 15 nucleotide bases, 16 nucleotide bases, 17 nucleotide bases, 18 nucleotide bases, 19 nucleotide bases, 20 nucleotide bases, 21 nucleotide bases, 22 nucleotide bases, 23 nucleotide bases, 24 nucleotide bases, 25 nucleotide bases, 26 nucleotide bases, 27 nucleotide bases, 28 nucleotide bases, 29 nucleotide bases, 30 nucleotide bases, 31 nucleotide bases, 32 nucleotide bases, 33 nucleotide bases, 34 nucleotide bases, 35 nucleotide bases, 37 nucleotide bases, 40 nucleotide bases, 42 nucleotide bases, 45 nucleotide bases, 47 nucleotide bases, or 50 nucleotide bases.

A primer may exhibit sequence identity or homology or complementarity to the template nucleic acid. The homology or sequence identity or complementarity between the primer and a template nucleic acid may be based on the length of the primer. For example, if the primer length is about 20 nucleic acids, it may contain 10 or more contiguous nucleic acid bases complementary to the template nucleic acid.

The term “primer extension reaction,” as used herein, generally refers to the binding of a primer to a strand of the template nucleic acid, followed by elongation of the primer(s). It may also include, denaturing of a double-stranded nucleic acid and the binding of a primer strand to either one or both of the denatured template nucleic acid strands, followed by elongation of the primer(s). Primer extension reactions may be used to incorporate nucleotides or nucleotide analogs to a primer in template-directed fashion by using enzymes (polymerizing enzymes).

The term “polymerase,” as used herein, generally refers to any enzyme capable of catalyzing a polymerization reaction. Examples of polymerases include, without limitation, a nucleic acid polymerase. The polymerase may be naturally occurring or synthesized. In some cases, a polymerase has relatively high processivity. An example polymerase is a Φ29 polymerase or a derivative thereof. A polymerase may be a polymerization enzyme. In some cases, a transcriptase or a ligase is used (i.e., enzymes which catalyze the formation of a bond). Examples of polymerases include a DNA polymerase, an RNA polymerase, a thermostable polymerase, a wild-type polymerase, a modified polymerase, E. coli DNA polymerase I, T7 DNA polymerase, bacteriophage T4 DNA polymerase Φ29 (phi29) DNA polymerase, Taq polymerase, Tth polymerase, Tli polymerase, Pfu polymerase, Pwo polymerase, VENT polymerase, DEEPVENT polymerase, EX-Taq polymerase, LA-Taq polymerase, Sso polymerase, Poc polymerase, Pab polymerase, Mth polymerase, ES4 polymerase, Tru polymerase, Tac polymerase, Tne polymerase, Tma polymerase, Tea polymerase, Tih polymerase, Tfi polymerase, Platinum Taq polymerases, Tbr polymerase, Tfl polymerase, Pfutubo polymerase, Pyrobest polymerase, Pwo polymerase, KOD polymerase, Bst polymerase, Sac polymerase, Klenow fragment, polymerase with 3′ to 5′ exonuclease activity, and variants, modified products and derivatives thereof. In some cases, the polymerase is a single subunit polymerase. The polymerase may have high processivity, namely the capability of the polymerase to consecutively incorporate nucleotides into a nucleic acid template without releasing the nucleic acid template. In some cases, a polymerase is a polymerase modified to accept dideoxynucleotide triphosphates, such as for example, Taq polymerase having a 667Y mutation (see e.g., Tabor et al, PNAS, 1995, 92, 6339-6343, which is herein incorporated by reference in its entirety for all purposes). In some cases, a polymerase is a polymerase having a modified nucleotide binding, which may be useful for nucleic acid sequencing, with non-limiting examples that include ThermoSequenas polymerase (GE Life Sciences), AmpliTaq FS (ThermoFisher) polymerase and Sequencing Pol polymerase (Jena Bioscience). In some cases, the polymerase is genetically engineered to have discrimination against dideoxynucleotides, such, as for example, Sequenase DNA polymerase (ThermoFisher).

The term “support,” as used herein, generally refers to a solid support such as a slide, a bead, a resin, a chip, an array, a matrix, a membrane, a nanopore, or a gel. The solid support may, for example, be a bead on a flat substrate (such as glass, plastic, silicon, etc.) or a bead within a well of a substrate. The substrate may have surface properties, such as textures, patterns, microstructure coatings, surfactants, or any combination thereof to retain the bead at a desire location (such as in a position to be in operative communication with a detector). The detector of bead-based supports may be configured to maintain substantially the same read rate independent of the size of the bead. The support may be a flow cell or an open substrate. Furthermore, the support may comprise a biological support, a non-biological support, an organic support, an inorganic support, or any combination thereof. The support may be in optical communication with the detector, may be physically in contact with the detector, may be separated from the detector by a distance, or any combination thereof. The support may have a plurality of independently addressable locations. The nucleic acid molecules may be immobilized to the support at a given independently addressable location of the plurality of independently addressable locations. Immobilization of each of the plurality of nucleic acid molecules to the support may be aided by the use of an adaptor. The support may be optically coupled to the detector. Immobilization on the support may be aided by an adaptor.

The term “label,” as used herein, generally refers to a moiety that is capable of coupling with a species, such as, for example, a nucleotide analog. In some cases, a label may be a detectable label that emits a signal (or reduces an already emitted signal) that can be detected. In some cases, such a signal may be indicative of incorporation of one or more nucleotides or nucleotide analogs. In some cases, a label may be coupled to a nucleotide or nucleotide analog, which nucleotide or nucleotide analog may be used in a primer extension reaction. In some cases, the label may be coupled to a nucleotide analog after the primer extension reaction. The label, in some cases, may be reactive specifically with a nucleotide or nucleotide analog. Coupling may be covalent or noncovalent (e.g., via ionic interactions, Van der Waals forces, etc.). In some cases, coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl)phosphine (TCEP)) or enzymatically cleavable (e.g., via an esterase, lipase, peptidase, or protease).

In some cases, the label may be optically active. In some embodiments, an optically-active label is an optically-active dye (e.g., fluorescent dye). Non-limiting examples of dyes include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5,, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), VIC, 5- (or 6-) iodoacetamidofluorescein, 5-{[2(and 3)-5-(Acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores.

In some examples, labels may be nucleic acid intercalator dyes. Examples include, but are not limited to ethidium bromide, YOYO-1, SYBR Green, and EvaGreen. The near-field interactions between energy donors and energy acceptors, between intercalators and energy donors, or between intercalators and energy acceptors may result in the generation of unique signals or a change in the signal amplitude. For example, such interactions may result in quenching (i.e., energy transfer from donor to acceptor that results in non-radiative energy decay) or Forster resonance energy transfer (FRET) (i.e., energy transfer from the donor to an acceptor that results in radiative energy decay). Other examples of labels include electrochemical labels, electrostatic labels, colorimetric labels and mass tags.

The term “quencher,” as used herein, generally refers to molecules that can reduce an emitted signal. Labels may be quencher molecules. For example, a template nucleic acid molecule may be designed to emit a detectable signal. Incorporation of a nucleotide or nucleotide analog comprising a quencher may reduce or eliminate the signal, which reduction or elimination is then detected. In some cases, as described elsewhere herein, labeling with a quencher may occur after nucleotide or nucleotide analog incorporation. Examples of quenchers include Black Hole Quencher Dyes (Biosearch Technologies) such as BH1-0, BHQ-1, BHQ-3, BHQ-10); QSY Dye fluorescent quenchers (from Molecular Probes/Invitrogen) such QSY7, QSY9, QSY21, QSY35, and other quenchers such as Dabcyl and Dabsyl; Cy5Q and Cy7Q and Dark Cyanine dyes (GE Healthcare). Examples of donor molecules whose signals may be reduced or eliminated in conjunction with the above quenchers include fluorophores such as Cy3B, Cy3, or Cy5; Dy-Quenchers (Dyomics), such as DYQ-660 and DYQ-661; fluorescein-5-maleimide; 7-diethylamino-3-(4′-maleimidylphenyl)-4-methylcoumarin (CPM); N-(7-dimethylamino-4-methylcoumarin-3-yl) maleimide (DACM) and ATTO fluorescent quenchers (ATTO-TEC GmbH), such as ATTO 540Q, 580Q, 612Q, 647N, Atto-633-iodoacetamide, tetramethylrhodamine iodoacetamide or Atto-488 iodoacetamide. In some cases, the label may be a type that does not self-quench for example, Bimane derivatives such as Monobromobimane.

The term “detector,” as used herein, generally refers to a device that is capable of detecting a signal, including a signal indicative of the presence or absence of an incorporated nucleotide or nucleotide analog. In some cases, a detector may include optical and/or electronic components that may detect signals. The term “detector” may be used in detection methods. Non-limiting examples of detection methods include optical detection, spectroscopic detection, electrostatic detection, electrochemical detection, and the like. Optical detection methods include, but are not limited to, fluorimetry and UV-vis light absorbance. Spectroscopic detection methods include, but are not limited to, mass spectrometry, nuclear magnetic resonance (NMR) spectroscopy, and infrared spectroscopy. Electrostatic detection methods include, but are not limited to, gel based techniques, such as, for example, gel electrophoresis. Electrochemical detection methods include, but are not limited to, electrochemical detection of amplified product after high-performance liquid chromatography separation of the amplified products.

The terms “sequence” or “sequence read,” as used herein, generally refer to a series of nucleotide assignments (e.g, by base calling) made during a sequencing process. Such sequences may be estimated sequence reads made by making preliminary base calls, which may then be subject to further base calling analysis or correction to produce final sequence reads. Sequences may comprise information corresponding to single or individual cells, and may be obtained by single-cell sequencing techniques (e.g., single-cell RNA sequencing, or scRNA-seq). Single-cell sequencing may be performed to provide a higher resolution of cellular differences and information about the function of an individual cell in the context of its microenvironment. For example, single-cell DNA sequencing can provide information about mutations present in rare cell populations (e.g., found in cancer cells), and single-cell RNA sequencing can provide information about individual cell expression corresponding to the existence and behavior of different cell types.

The terms “single guide RNA” or “sgRNA,” as used herein, generally refer to a single RNA molecule that contains both a custom-designed short CRISPR RNA (crRNA) sequence fused to a scaffold trans-activating crRNA (tracrRNA) sequence. The sgRNA can be synthetically generated or made in vitro or in vivo from a DNA template.

The term “drug,” as used herein, generally refers to a biological or chemical substance that causes a biological effect in a subject when consumed. A drug may comprise a chemical substance which, when administered to a subject, produces a biological effect in the subject. A drug may be used to treat a given target indication, such as a disease. For example, the drug may be a pharmaceutical drug (e.g., a medication or medicine) used to treat, cure, or prevent a disease or promote well-being. The disease may be cancer, acne, attention deficit hyperactivity disorder, AIDS/HIV, allergies, Alzheimer’s, angina, anxiety, arthritis, asthma, bipolar disorder, bronchitis, hypercholesterolemia, cold or flu, constipation, chronic obstructive pulmonary disorder, Covid-19, depression, diabetes, eczema, erectile dysfunction, fibromyalgia, gastrointestinal, heartburn, gout, heart disease, herpes, hypertension, hypothyroidism, irritable bowel disease, incontinence, migraine, osteoarthritis, pneumonia, psoriasis, rheumatoid arthritis, schizophrenia, seizures, stroke, swine flu, or urinary tract infection. The drug may be administered via ingestion, inhalation, injection, smoking, topical application, absorption via a patch on the skin, suppository, or dissolution under the tongue. The drug may comprise a pharmaceutical, a compound (e.g., small molecule), an inhibitor (e.g., small molecular inhibitor), an antibody, an siRNA, an antisense oligonucleotide, an mRNA therapy, or a combination thereof.

The term “effectiveness,” as used herein, generally refers to an expected or average efficacy of a drug (e.g., across a population of subjects). The efficacy may be a maximum response achievable from a dose of a drug that is administered to a subject. In some examples, effectiveness may be determined for a drug that binds to a target gene, as a degree to which the function of the bound target gene is affected. For example, if a drug inhibits a particular target gene upon binding to the target gene, the drug has on-target gene inhibition effects, which may be measured by the relative decrease in gene expression levels of the target gene. As another example, a drug may be determined to have a high effectiveness for a particular target based on a measured transcriptome having maximal similarity to an on-target reference transcriptome and/or a minimal similarity to an off-target reference transcriptome. As another example, a drug may be determined to have a low effectiveness for a particular target based on a measured transcriptome having low similarity to an on-target reference transcriptome and/or a high similarity to an off-target reference transcriptome.

The ability to selectively modify target genomic regions of cells to alter their cellular states (e.g., by converting cells from one differentiated state to another) may hold great promise for therapeutic applications. However, despite the promise of selective modification of cellular states (e.g., via cellular re-programming), the identification of genetic drivers that may mediate the transition between one cell state to another remains challenging for many therapeutically relevant applications. For example, the phenotype of re-programming may be complex and may involve many genes interacting with each other in a hierarchical, non-linear fashion. Disentangling which of these genes is causal versus correlative in a given process may be a challenging task and may require extensive, time-intensive experimental assays and animal models for each gene of interest. Further, therapeutic targeting using drugs, such as treatment inhibitors, may be evaluated for effectiveness in subjects with a disease or disorder.

Recognized herein is a need for improved methods for determining an effectiveness of a drug. Such drugs may be associated with certain genomic regions that are suitable for therapeutic targeting (e.g., genomic regions which may facilitate re-programming of a cell from one phenotypic state to another). Methods and systems provided herein may significantly increase the efficiency, accuracy, and/or throughput of determining the effectiveness of drugs. Such methods and systems may leverage the identification of certain genomic regions for therapeutic targeting.

The present disclosure relates generally to methods and systems for determining an effectiveness of a drug. Such drugs may be associated with target genomic regions of cells that may be selectively modified to alter their cellular states (e.g., via transcriptional re-programming of cells from one differentiated state to another). For example, the present technology relates to high-throughput screening of drug candidates, which may leverage high-content, high-efficiency, and high-throughput CRISPR (clustered regularly interspaced short palindromic repeats) screening techniques for identifying relevant target genes that may potentially mediate re-programming between phenotypically distinct cellular states and/or be selected as effective therapeutic targets. These screens may leverage anomaly detection models to quantify re-programming as a measurable phenotype for each gene that is targeted via CRISPR. Methods and systems of the present disclosure may effectively determine an effectiveness of a drug, based at least in part on quantification of the ability to selectively modify target genomic regions of cells (e.g., via cellular re-programming) as a basis for choosing biomarkers and therapeutic targets relevant to a disease indication of interest.

In an aspect, the present disclosure provides a method for determining an effectiveness of a drug, comprising: (a) generating a latent space representation of nucleic acid sequence data for a plurality of diseased cells and a plurality of normal cells of a cell type, wherein said latent space represents a plurality of phenotypic states of said cell type; (b) identifying, based at least in part on a topology of said latent space, a genomic region that facilitates reprogramming of said cell type from a first phenotypic state to a second phenotypic state of said plurality of phenotypic states; (c) mapping sequence data of a first cell of said cell type to said latent space to yield a first latent space representation, wherein said first cell has been reprogrammed from said first phenotypic state to said second phenotypic state; (d) mapping sequence data of a second cell of said cell type to said latent space to yield a second latent space representation, wherein said second cell has been exposed to said drug, and wherein prior to said second cell being exposed to said drug, said second cell exhibited said first phenotypic state; and (e) determining, based at least in part on said first latent space representation and said second latent space representation, said effectiveness of said drug.

In some embodiments, (a) comprises using a supervised dimensionality reduction algorithm to generate said latent space representation. In some embodiments, said supervised dimensionality reduction algorithm is a uniform manifold approximation and projection (UMAP) algorithm. In some embodiments, said supervised dimensionality reduction algorithm is a t-distributed stochastic neighbor embedding (t-SNE) algorithm. In some embodiments, said supervised dimensionality reduction algorithm is a variable autoencoder. In some embodiments, (b) comprises performing non-linear cell trajectory reconstruction on said latent space to construct an inferred maximum likelihood progression trajectory between said first phenotypic state and said second phenotypic state. In some embodiments, performing said non-linear cell trajectory reconstruction comprises applying a reverse graph embedding algorithm to said latent space.

In some embodiments, said first phenotypic state is cancer and said second phenotypic state is a wildtype state. In some embodiments, said second phenotypic state is an intermediate state. In some embodiments, said intermediate state is a fibroblast state or a progenitor cell state. In some embodiments, said first cell has been reprogrammed from said first phenotypic state to said second phenotypic state using genetic editing. In some embodiments, said genetic editing is performed with a genetic editing unit selected from the group consisting of a CRISPR (e.g., active Cas9) system, a CRISPRi (e.g., CRISPR interference, a catalytically dead Cas9 fused to a transcriptional repressor peptide including KRAB) system, a CRISPRa (e.g., CRISPR activation, a catalytically dead Cas9 fused to a transcriptional activator peptide including VPR (HIV viral protein R)) system, a RNAi system, and a shRNA system

In some embodiments, (e) comprises measuring (i) a shift in said latent space representation of said first cell from said editing and (ii) a shift in said latent space representation of said second cell from said exposure to said drug; and mathematically relating (i) to (ii). In some embodiments, said measuring comprises using a supervised learning algorithm. In some embodiments, said supervised learning algorithm is a support vector machine, a random forest, logistic regression, a Bayesian classifier, or a convolutional neural network.

In some embodiments, the method further comprises: mapping nucleic acid sequence data of a plurality of additional cells of said cell type to said latent space, wherein each cell of said plurality of additional cells has been exposed to a respective drug of a plurality of drugs; determining, based at least in part on said latent space representation of said first cell and latent space representations of said plurality of additional cells, an effectiveness of each drug; and electronically outputting a ranking of said plurality of drugs based at least in part on said effectiveness of each drug. In some embodiments, said drug is selected from the group consisting of: a compound (e.g., a small molecule), an inhibitor (e.g., a small molecule inhibitor), and an antibody.

In some embodiments, at least one of said sequence data of said first cell of said cell type and said sequence data of said second cell of said cell type is generated by single-cell sequencing. In some embodiments, at least one of said sequence data of said first cell of said cell type and said sequence data of said second cell of said cell type is generated by sequential single-cell sequencing.

In another aspect, the present disclosure provides a method for determining an effectiveness of a drug, comprising: (a) generating a latent space representation of nucleic acid sequence data for a plurality of diseased cells and a plurality of normal cells of a cell type, wherein said latent space represents a plurality of phenotypic states of said cell type; (b) identifying, based at least in part on a topology of said latent space, a target genomic region of said cell type; (c) mapping sequence data of a first cell of said cell type to said latent space to yield a first latent space representation, wherein said target genomic region of said first cell has been modified, and wherein said first cell exhibited a first phenotypic state prior to said modification; (d) mapping sequence data of a second cell of said cell type to said latent space to yield a second latent space representation, wherein said second cell has been exposed to said drug, and wherein prior to said second cell being exposed to said drug, said second cell exhibited said first phenotypic state; and (e) determining, based at least in part on said first latent space representation and said second latent space representation, said effectiveness of said drug.

In some embodiments, (a) comprises using a supervised dimensionality reduction algorithm to generate said latent space representation. In some embodiments, said supervised dimensionality reduction algorithm is a uniform manifold approximation and projection (UMAP) algorithm. In some embodiments, said supervised dimensionality reduction algorithm is a t-distributed stochastic neighbor embedding (t-SNE) algorithm. In some embodiments, said supervised dimensionality reduction algorithm is a variable autoencoder.

In some embodiments, said first phenotypic state is cancer. In some embodiments, said first phenotypic state is an intermediate state. In some embodiments, said intermediate state is a fibroblast state or a progenitor cell state.

In some embodiments, (e) comprises measuring (i) a shift in said latent space representation of said first cell from said modification, and (ii) a shift in said latent space representation of said second cell from said exposure to said drug; and mathematically relating (i) to (ii). In some embodiments, said measuring comprises using a supervised learning algorithm. In some embodiments, said supervised learning algorithm is a support vector machine, a random forest, logistic regression, a Bayesian classifier, or a convolutional neural network.

In some embodiments, the method further comprises: mapping nucleic acid sequence data of a plurality of additional cells of said cell type to said latent space, wherein each cell of said plurality of additional cells has been exposed to a respective drug of a plurality of drugs; determining, based at least in part on said latent space representation of said first cell and latent space representations of said plurality of additional cells, an effectiveness of each drug; and electronically outputting a ranking of said plurality of drugs based at least in part on said effectiveness of each drug. In some embodiments, said drug is selected from the group consisting of: a compound (e.g., a small molecule), an inhibitor (e.g., a small molecule inhibitor), and an antibody.

In some embodiments, at least one of said sequence data of said first cell of said cell type and said sequence data of said second cell of said cell type is generated by single-cell sequencing. In some embodiments, at least one of said sequence data of said first cell of said cell type and said sequence data of said second cell of said cell type is generated by sequential single-cell sequencing.

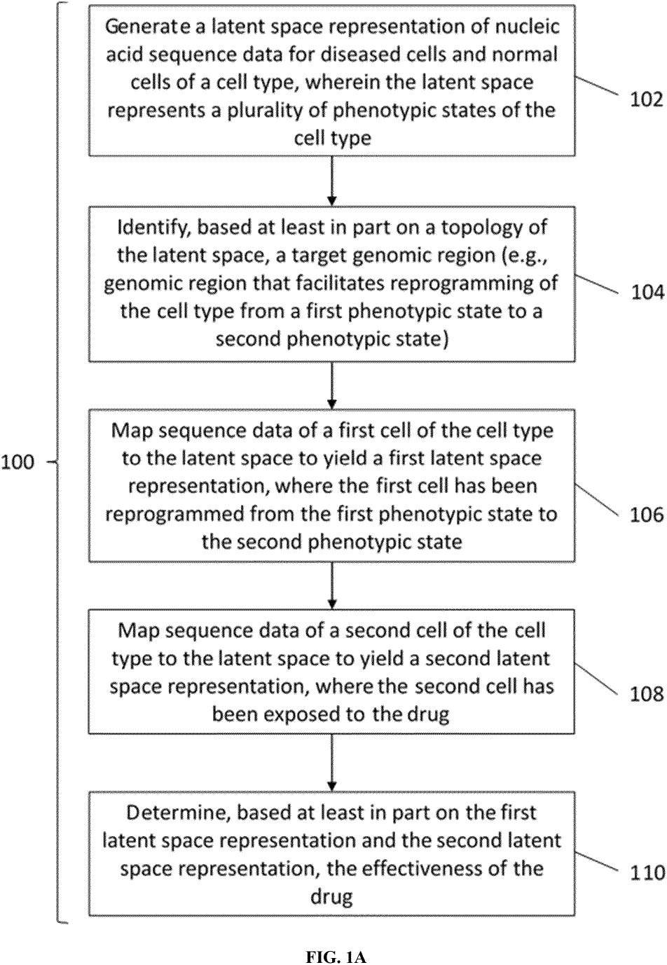

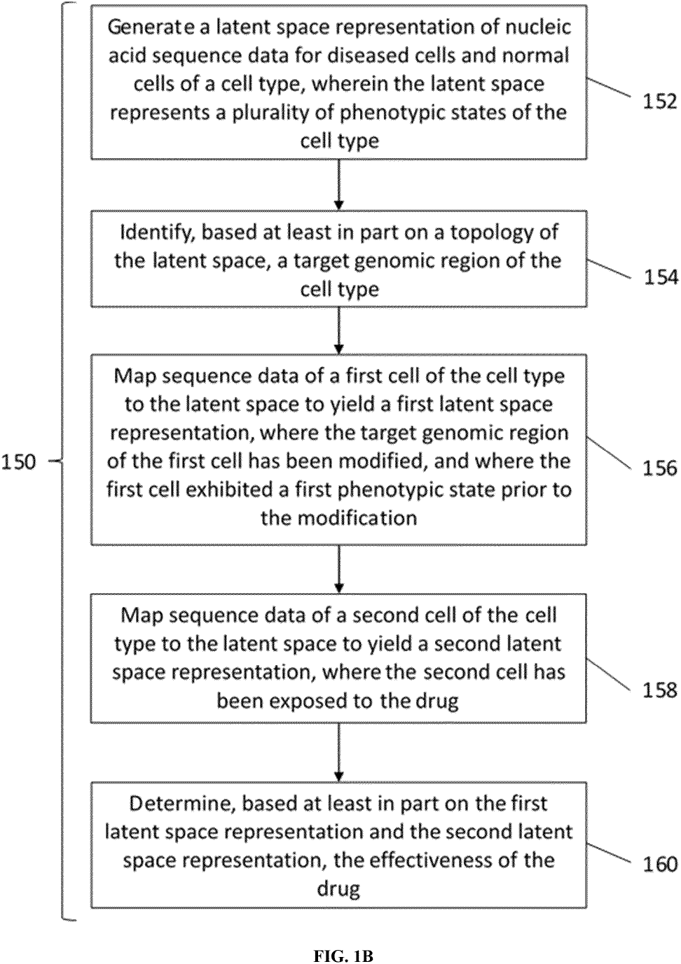

FIG. 1A shows an example of a flowchart illustrating a method 100 for determining an effectiveness of a drug. The method may comprise generating a latent space representation of nucleic acid sequence data for a plurality of diseased cells and a plurality of normal cells of a cell type (as in operation 102). For example, in some embodiments, the latent space represents a plurality of phenotypic states of the cell type. Next, the method may comprise identifying a target genomic region (e.g., a genomic region that facilitates reprogramming of the cell type from a first phenotypic state to a second phenotypic state of the plurality of phenotypic states) (as in operation 104). For example, in some embodiments, the target genomic region is identified based at least in part on a topology of the latent space. Next, the method may comprise mapping sequence data of a first cell of the cell type to the latent space to yield a first latent space representation (as in operation 106). For example, in some embodiments, the first cell has been reprogrammed from the first phenotypic state to the second phenotypic state. Next, the method may comprise mapping sequence data of a second cell of the cell type to the latent space to yield a second latent space representation (as in operation 108). For example, in some embodiments, the second cell has been exposed to the drug. In some embodiments, prior to the second cell being exposed to the drug, the second cell exhibited the first phenotypic state. Next, the method may comprise determining the effectiveness of the drug (as in operation 110). For example, in some embodiments, the effectiveness of the drug is determined based at least in part on the first latent space representation and the second latent space representation.

FIG. 1B shows another example of a flowchart illustrating a method 150 for determining an effectiveness of a drug. The method may comprise generating a latent space representation of nucleic acid sequence data for a plurality of diseased cells and a plurality of normal cells of a cell type (as in operation 152). For example, in some embodiments, the latent space represents a plurality of phenotypic states of the cell type. Next, the method may comprise identifying a target genomic region of the cell type (as in operation 154). Next, the method may comprise mapping sequence data of a first cell of the cell type to the latent space to yield a first latent space representation (as in operation 156). For example, in some embodiments, the target genomic region of the first cell has been modified. For example, in some embodiments, the first cell exhibited a first phenotypic state prior to the modification. Next, the method may comprise mapping sequence data of a second cell of the cell type to the latent space to yield a second latent space representation (as in operation 158). For example, in some embodiments, the second cell has been exposed to the drug. In some embodiments, prior to the second cell being exposed to the drug, the second cell exhibited the first phenotypic state. Next, the method may comprise determining the effectiveness of the drug (as in operation 160). For example, in some embodiments, the effectiveness of the drug is determined based at least in part on the first latent space representation and the second latent space representation.

In some embodiments, the UMAP algorithm is a supervised UMAP algorithm or an unsupervised supervised UMAP algorithm. For example, a supervised UMAP algorithm may be trained on a dataset comprising single-cell RNA sequence (scRNA-seq) data of pure cells of a given cell type. The UMAP algorithm may be trained using a minimum distance of about 0.025, about 0.05, about 0.075, about 0.1, about 0.125, about 0.15, about 0.175, about 0.2, about 0.225, about 0.25, about 0.275, about 0.3, about 0.325, about 0.35, about 0.375, about 0.4, about 0.425, about 0.45, about 0.475, about 0.5, about 0.525, about 0.55, about 0.575, about 0.6, about 0.625, about 0.65, about 0.675, about 0.7, about 0.725, about 0.75, about 0.775, about 0.8, about 0.825, about 0.85, about 0.875, about 0.9, about 0.925, about 0.95, about 0.975, or about 1.0. In some embodiments, prior to the mapping, low-frequency genomic regions may be removed from the single-cell RNA sequence (scRNA-seq) data for the plurality of diseased cells and the plurality of normal cells.

The identification of the one or more genomic regions that facilitate re-programming of the cell type between the first phenotypic state and the second phenotypic state may be performed based at any of a number of suitable analyses of a topology of the latent space. As an example, non-linear cell trajectory reconstruction may be conducted on the latent space (e.g., by applying the reverse graph embedding algorithm to the latent space) to construct an inferred maximum likelihood progression trajectory between the first phenotypic state and the second phenotypic state. Then, based on the inferred maximum likelihood progression trajectory, probabilistic inference may be used to identify the one or more genomic regions that facilitate re-programming of the cell type between the first phenotypic state and the second phenotypic state. In some embodiments, one or more therapeutic targets may be identified to treat a disease associated with the first phenotypic state, based on the identified genomic regions.

After the genomic regions are identified, a genomic editing unit (e.g., a CRISPR (e.g., active Cas9) system, a CRISPRi (e.g., CRISPR interference, a catalytically dead Cas9 fused to a transcriptional repressor peptide including KRAB) system, CRISPRa (e.g., CRISPR activation, a catalytically dead Cas9 fused to a transcriptional activator peptide including VPR (HIV viral protein R)) system, an RNAi system, or an shRNA system) may be used to edit a respective genomic region to facilitate the re-programming of a cell of the cell type between the first phenotypic state and the second phenotypic state. After the editing, an anomaly detection algorithm may be used to measure a quantity of a shift in the latent space of the cell as a result of using the genomic editing unit to edit the respective genomic region (e.g., using a density estimation function). For example, the quantity of the shift in the latent space may be measured using a distance measure (e.g., a Chebychev distance, a Correlation distance, a Cosine distance, a Euclidean distance, a signed Euclidean distance, a Hamming distance, a Jaccard distance, a Kullback-Leibler distance, a Mahalanobis distance, a Manhattan distance, a Minkowski distance, a Spearman distance, or a distance on a Riemannian manifold). For example, the density estimation function may comprise a probability density estimation, a rescaled histogram, a parametric density estimation function, a non-parametric density estimation function (e.g., a kernel density function), or a data clustering technique (e.g., vector quantization).

The anomaly detection algorithm may comprise an unsupervised machine learning algorithm, a semi-supervised machine learning algorithm, or a supervised machine learning algorithm, which may be trained on latent space profiles of a plurality of cell types, such as diseased cell types (e.g., cancer cells such as pancreatic cancer cells) or non-diseased cell types (e.g., pancreatic cells such as pancreatic ductal or acinar cells). For example, the anomaly detection algorithm may comprise one or more of: a density-based technique (k-nearest neighbor, local outlier factor, isolation forest), a subspace-based outlier detection, a correlation-based outlier detection, a tensor-based outlier detection, a support vector machine (SVM), a single-class vector machine, support vector data description, a neural network (e.g., replicator neural network, autoencoder, long short-term memory (LSTM) neural network), a Bayesian network, a hidden Markov model (HMM), a cluster analysis-based outlier detection, deviation from association rules and frequent itemsets, fuzzy logic-based outlier detection, and an ensemble technique (e.g., using feature bagging, score normalization, and different sources of diversity). The diseased cells or normal cells may comprise, for example, primary cell lines, human organoids, and animal models. For example, the plurality of cell types may include pancreatic ductal cells, pancreatic acinar cells, pancreatic adenocarcinomas, and/or pancreatic adenocarcinomas. After measuring the quantities of shifts in the latent space of the cell as a result of using the genomic editing unit to edit the respective genomic region, the one or more genes may be ranked for therapeutic targeting based on the measured quantities.

In another aspect, the present disclosure provides a non-transitory computer-readable medium comprising machine-executable code that, upon execution by one or more computer processors, implements a method for determining an effectiveness of a drug, said method comprising: (a) generating a latent space representation of nucleic acid sequence data for a plurality of diseased cells and a plurality of normal cells of a cell type, wherein said latent space represents a plurality of phenotypic states of said cell type; (b) identifying, based at least in part on a topology of said latent space, a genomic region that facilitates reprogramming of said cell type from a first phenotypic state to a second phenotypic state of said plurality of phenotypic states; (c) mapping sequence data of a first cell of said cell type to said latent space to yield a first latent space representation, wherein said first cell has been reprogrammed from said first phenotypic state to said second phenotypic state; (d) mapping sequence data of a second cell of said cell type to said latent space to yield a second latent space representation, wherein said second cell has been exposed to said drug, and wherein prior to said second cell being exposed to said drug, said second cell exhibited said first phenotypic state; and (e) determining, based at least in part on said first latent space representation and said second latent space representation, said effectiveness of said drug.

In another aspect, the present disclosure provides a system for determining an effectiveness of a drug, comprising: a database that comprises nucleic acid sequence data for a plurality of diseased cells and a plurality of normal cells of a cell type; and one or more computer processors that are individually or collectively programmed to: (i) generate a latent space representation of said nucleic acid sequence data, wherein said latent space represents a plurality of phenotypic states of said cell type; (ii) identify, based at least in part on a topology of said latent space, a target genomic region of said cell type; (iii) map sequence data of a first cell of said cell type to said latent space to yield a first latent space representation, wherein said target genomic region of said first cell has been modified, and wherein said first cell exhibited a first phenotypic state prior to said modification; (iv) map sequence data of a second cell of said cell type to said latent space to yield a second latent space representation, wherein said second cell has been exposed to said drug, and wherein prior to said second cell being exposed to said drug, said second cell exhibited said first phenotypic state; and (v) determine, based at least in part on said first latent space representation and said second latent space representation, said effectiveness of said drug.

In another aspect, the present disclosure provides a non-transitory computer-readable medium comprising machine-executable code that, upon execution by one or more computer processors, implements a method for determining an effectiveness of a drug, said method comprising: (a) generating a latent space representation of nucleic acid sequence data for a plurality of diseased cells and a plurality of normal cells of a cell type, wherein said latent space represents a plurality of phenotypic states of said cell type; (b) identifying, based at least in part on a topology of said latent space, a target genomic region of said cell type; (c) mapping sequence data of a first cell of said cell type to said latent space to yield a first latent space representation, wherein said target genomic region of said first cell has been modified, and wherein said first cell exhibited a first phenotypic state prior to said modification; (d) mapping sequence data of a second cell of said cell type to said latent space to yield a second latent space representation, wherein said second cell has been exposed to said drug, and wherein prior to said second cell being exposed to said drug, said second cell exhibited said first phenotypic state; and (e) determining, based at least in part on said first latent space representation and said second latent space representation, said effectiveness of said drug.

In another aspect, the present disclosure provides a system for identifying one or more genomic regions that facilitate re-programming of a cell from one phenotypic state to another. The system may comprise a database that comprises single-cell RNA sequence data (e.g., for a plurality of diseased cells and a plurality of normal cells of a cell type). The database may be stored locally (e.g., on a local server, computer, or computer media) or remotely (e.g., a cloud-based server). The system may further comprise one or more computer processors that are individually or collectively programmed to implement methods of the present disclosure. For example, the computer processors may be individually or collectively programmed to perform one or more of: mapping (e.g., using a UMAP algorithm or a supervised dimensionality reduction algorithm) the single-cell RNA sequence (scRNA-seq) data for the plurality of diseased cells and the plurality of normal cells into a latent space corresponding to a plurality of phenotypic states of the cell type; identifying, based at least in part on a topology of the latent space, the one or more genomic regions that facilitate reprogramming of the cell type between a first phenotypic state and a second phenotypic state of the plurality of phenotypic states (e.g., wherein the one or more genomic regions are configured to be edited to facilitate the re-programming of the cell type between the first phenotypic state and the second phenotypic state); and/or electronically outputting the one or more genomic regions.

In another aspect, the present disclosure provides a system for determining an effectiveness of a drug, comprising: a database that comprises nucleic acid sequence data for a plurality of diseased cells and a plurality of normal cells of a cell type; and one or more computer processors that are individually or collectively programmed to: (i) generate a latent space representation of said nucleic acid sequence data, wherein said latent space represents a plurality of phenotypic states of said cell type; (ii) identify, based at least in part on a topology of said latent space, a genomic region that facilitates reprogramming of said cell type from a first phenotypic state to a second phenotypic state of said plurality of phenotypic states; (iii) map sequence data of a first cell of said cell type to said latent space to yield a first latent space representation, wherein said first cell has been reprogrammed from said first phenotypic state to said second phenotypic state; (iv) map sequence data of a second cell of said cell type to said latent space to yield a second latent space representation, wherein said second cell has been exposed to said drug, and wherein prior to said second cell being exposed to said drug, said second cell exhibited said first phenotypic state; and (v) determine, based at least in part on said first latent space representation and said second latent space representation, said effectiveness of said drug.

### Computer Systems

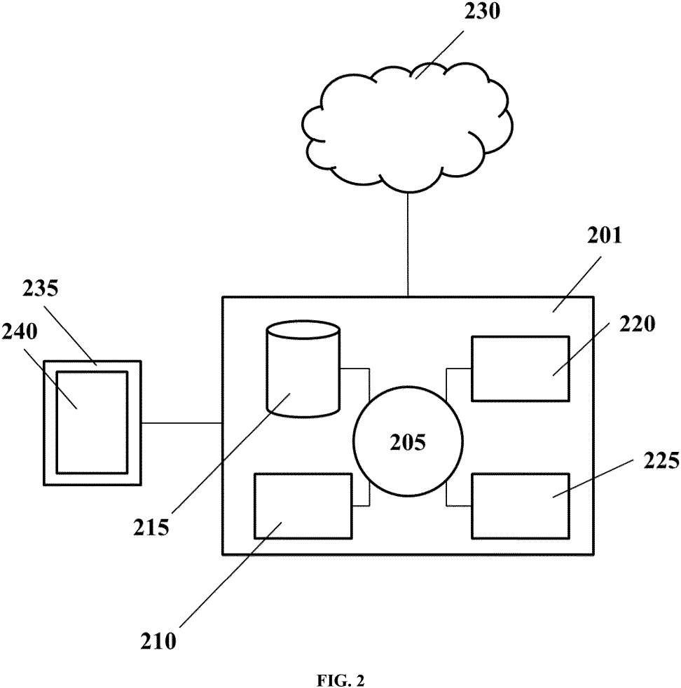

The present disclosure provides computer systems that are programmed to implement methods of the disclosure. FIG. 2 shows a computer system 201 that is programmed or otherwise configured to, for example: generate or analyze nucleic acid sequence data (e.g., scRNA-seq data), generate a latent space representation of nucleic acid data, map sequence data to a latent space, identify target genomic regions (e.g., genomic regions that facilitate re-programming of a cell type between a first phenotypic state and a second phenotypic state) (e.g., using probabilistic inference), train a supervised algorithm on nucleic acid sequence data, and determine the effectiveness of drugs.

The computer system 201 can regulate various aspects of methods and systems of the present disclosure, such as, for example, generating or analyzing nucleic acid sequence data (e.g., scRNA-seq data), generate a latent space representation of nucleic acid data, mapping sequence data to a latent space, identifying target genomic regions (e.g., genomic regions that facilitate reprogramming of a cell type between a first phenotypic state and a second phenotypic state) (e.g., using probabilistic inference), training a supervised algorithm on nucleic acid sequence data, and determining the effectiveness of drugs.

The computer system 201 can be an electronic device of a user or a computer system that is remotely located with respect to the electronic device. The electronic device can be a mobile electronic device. The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220 and peripheral devices 225 are in communication with the CPU 205 through a communication bus (solid lines), such as a motherboard. The storage unit 215 can be a data storage unit (or data repository) for storing data. The computer system 201 can be operatively coupled to a computer network (“network”) 230 with the aid of the communication interface 220. The network 230 can be the Internet, an internet and/or extranet, or an intranet and/or extranet that is in communication with the Internet. The network 230 in some cases is a telecommunication and/or data network. The network 230 can include one or more computer servers, which can enable distributed computing, such as cloud computing. The network 230, in some cases with the aid of the computer system 201, can implement a peer-to-peer network, which may enable devices coupled to the computer system 201 to behave as a client or a server.

The CPU 205 can execute a sequence of machine-readable instructions, which can be embodied in a program or software. The instructions may be stored in a memory location, such as the memory 210. The instructions can be directed to the CPU 205, which can subsequently program or otherwise configure the CPU 205 to implement methods of the present disclosure. Examples of operations performed by the CPU 205 can include fetch, decode, execute, and writeback.

The CPU 205 can be part of a circuit, such as an integrated circuit. One or more other components of the system 201 can be included in the circuit. In some cases, the circuit is an application specific integrated circuit (ASIC).

The storage unit 215 can store files, such as drivers, libraries and saved programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet.

The computer system 201 can communicate with one or more remote computer systems through the network 230. For instance, the computer system 201 can communicate with a remote computer system of a user. Examples of remote computer systems include personal computers (e.g., portable PC), slate or tablet PC’s (e.g., Apple^{®} iPad, Samsung^{®} Galaxy Tab), telephones, Smart phones (e.g., Apple^{®} iPhone, Android-enabled device, Blackberry^{®}), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine-executable or machine-readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer-readable media may be involved in carrying one or more sequences of one or more instructions to a processor for execution.

The computer system 201 can include or be in communication with an electronic display 235 that comprises a user interface (UI) 240 for providing, for example, user selection of nucleic acid sequence data, mapping or other algorithms, and databases. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 205. The algorithm can, for example, generate or analyze nucleic acid sequence data (e.g., scRNA-seq data), generate a latent space representation of nucleic acid data, map sequence data to a latent space, identify target genomic regions (e.g., genomic regions that facilitate re-programming of a cell type between a first phenotypic state and a second phenotypic state) (e.g., using probabilistic inference), train a supervised algorithm on nucleic acid sequence data, and determine the effectiveness of drugs.

### EXAMPLES

### Example 1 - Generation and Pre-Processing of scRNA-seq Data

Single-cell RNA sequencing (scRNA-seq) data were generated as follows. Cells from the human KRAS-mutant (KRAS^{G12C}) cancer pancreatic cancer cell line MIAPaCa-2 and the normal pancreatic duct cell line hTERT-HPNE (Human Pancreatic Nestin Expressing cells) were cultured in DMEM media, supplemented with FBS, and additional components according to the vendor’s instructions. For pharmacological inhibition, these cell lines were treated with one of various small-molecule inhibitors, including Auranofin, D9, and Piperlongumine. For genetic inhibition, these cell lines were further genetically modified to stably express a catalytically dead Cas9 (dCas9) fused to a transcriptional repressor peptide Kruppel associated box (KRAB), enabling CRISPR interference (CRISPRi) for silencing genes of interest by co-expressing an sgRNA targeting KRAS, TXNRD1, or RPA1 individually. For scRNA-seq, each type of cells was single-cell isolated, and then their corresponding RNA and cDNA libraries were prepared according to the manufacturer’s instructions (10X Genomics, Pleasanton, CA). cDNA libraries were sequenced by MiSeq sequencing instruments (Illumina, San Diego, CA) to acquire cell number information, and then sequenced by NextSeq instruments (Illumina) or Hiseq4000 instruments (Illumina) to acquire scRNA-seq data.

Single-cell RNA sequencing (scRNA-seq) data were pre-processed as follows. The raw, HUGO Gene Nomenclature Committee (HGNC)-aligned, unique molecular index (UMI) count matrix generated via 10X depth sequencing was preprocessed and scaled prior to analyzing in downstream analysis pipelines. Low-abundance genes (e.g. having an average count of less than 0.1) and genes with reads in less than 10% of cells, as well as cells with non-zero reads for less than 10% of all genes, were removed from the count matrix. To adjust for discrepancies in sequencing depth between individual cells, count matrices were, in some cases, normalized and scaled prior to carrying forward in subsequent analyses. Methods of normalization include, but are not limited to: globally scaling cell-level counts to the median depth or the mean depth across all cells (scalar adjustment), deconvolution approaches such as solving linear systems to obtain unique scaling factors for individual cells, scaling normalization using summed values across pools of cells, and scaling normalization using spike-in RNA sets. In some cases, inter-sample batch effects were corrected via a mutual nearest neighbors algorithm (MNN), a principal component analysis (PCA), a multi-batch normalization, a multi-batch PCA, etc.

### Example 2 - Latent Space Construction

Latent space construction was performed as follows. The high-dimensional, single-cell count matrix was mapped to a 2-dimensional latent space using supervised machine learning algorithms. In the case of pancreatic cancer, the reduction algorithm was trained on a collection of pure cell types, including pancreatic acinar, ductal, and adenocarcinoma cells. Cells targeted with an essential gene (e.g. RPA1 or PCNA) were also included during latent space training in order to model potential toxicity complications that may arise from a target candidate of interest. The labels for supervised learning were chosen to correspond to each of the pure cell types.

Several algorithms were evaluated for latent space construction, including but not limited to: uniform manifold approximation and projection (UMAP) as well as variable autoencoders (VAEs). In some cases, the Elbow method (e.g., as described by Richards et al., J Shoulder Elbow Surg 8(4): 351-354 (1999), which is incorporated by reference herein in its entirety) was used to determine the optimal number of dimensions for the latent space. For UMAP, the following parameters were used for model training: a minimum distance of 0.025-0.25, a number of neighbors equal to 75% of the total number of cells, and a Euclidean distance as the distance metric.

### Example 3 - Drug Treatment Quantification and Selection

Drug treatment effects were quantified based on the relative conversion of cells from a diseased state to a target state following drug treatment. Briefly, a supervised classification algorithm was trained on 2-dimensional latent expression profiles of pure cell types described above, including diseased cells (e.g., cancer) and target (e.g., primary) cells. The algorithm was trained to discriminate between cell types in a binary fashion. Examples of algorithms included but were not limited to: Random Forests, Logistic Regression, Bayesian classifiers, convolutional neural networks, and support vector machines. Objective functions for the algorithms were optimized such that they were able to discriminate between cell types with a bootstrapped-averaged area-under-the-curve (AUC) exceeding 0.98.

Diseased cells (e.g., cancer cells) were then treated with candidate drug compounds for a set duration (e.g., 6 hours or 24 hours), and drug-treated cells were assigned as “diseased” or “target” cells via the trained classifier described above. The proportion of drug-treated cells that were successfully “converted” to the “target” state on the basis of this classification output was then evaluated against a vehicle control treatment, such as DMSO. A 95% confidence interval for the proportion was constructed via iterative sampling with replacement. Drugs were then ranked based on effect size (relative to the vehicle control) or mean bootstrapped proportion. Top drug candidates satisfying a Bonferroni-adjusted p-value of < 0.05 were selected as putative compounds for further biological studies and development.

### Example 4 - A Pipeline for Comparing Effects From Genetic and Pharmacological Inhibitions and Identifying On-Target Inhibitors

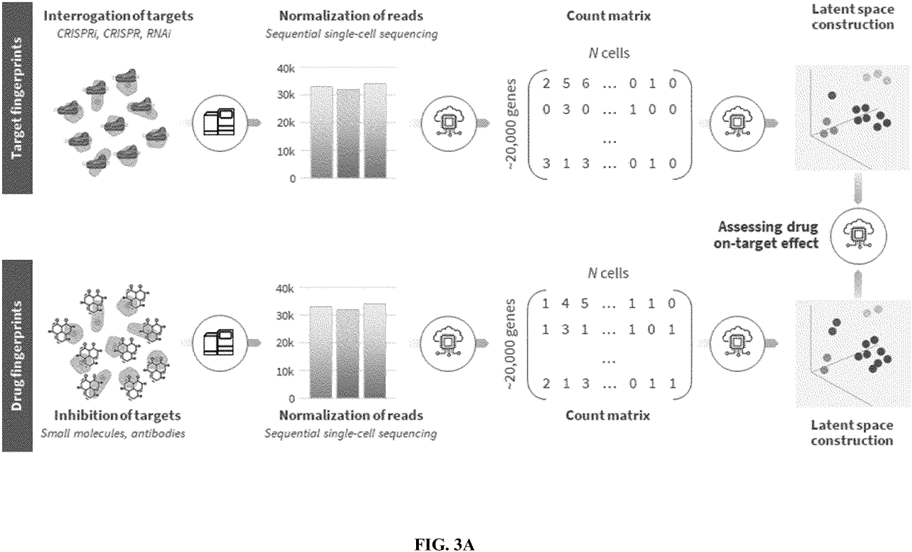

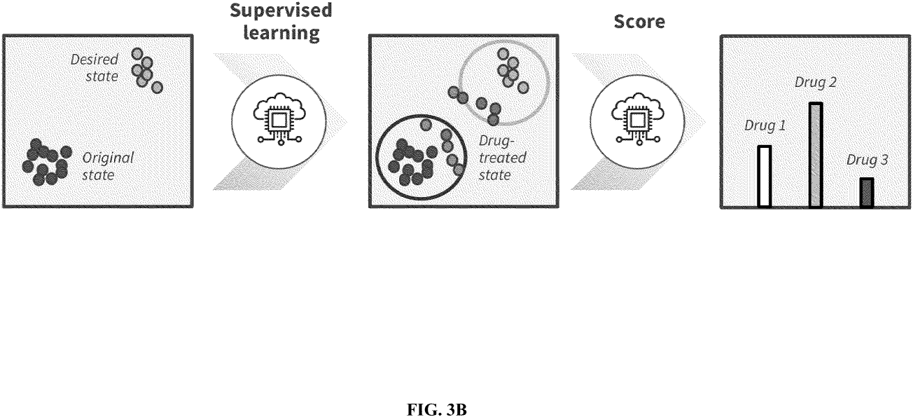

FIGS. 3A-3B provide an experimental and computational framework for identifying inhibitors that best mimic the effect of gene interrogation by CRISPRi (or CRISPR, RNAi). FIG. 3A shows an example of assessing drugs’ on- and off-target effects and identification of novel inhibitors. By leveraging CRISPRi gene interrogation, sequential single-cell sequencing, intelligent latent space construction, and supervised learning, on- and off-target effects from drug fingerprints (inhibition of targets by small molecules, antibodies) are assessed in accordance with their ability to match a desired state dictated by target fingerprints (interrogation of targets by CRISPRi, CRISPR, RNAi). For example, performing sequential single-cell sequencing advantageously increases the robustness of the analysis and decreases undesirable effects (e.g., batch effects and/or background noise).

FIG. 3B shows an illustration of supervised learning as a method for training model on binary cell types to classify new cells by comparing classifications with original and desired states.

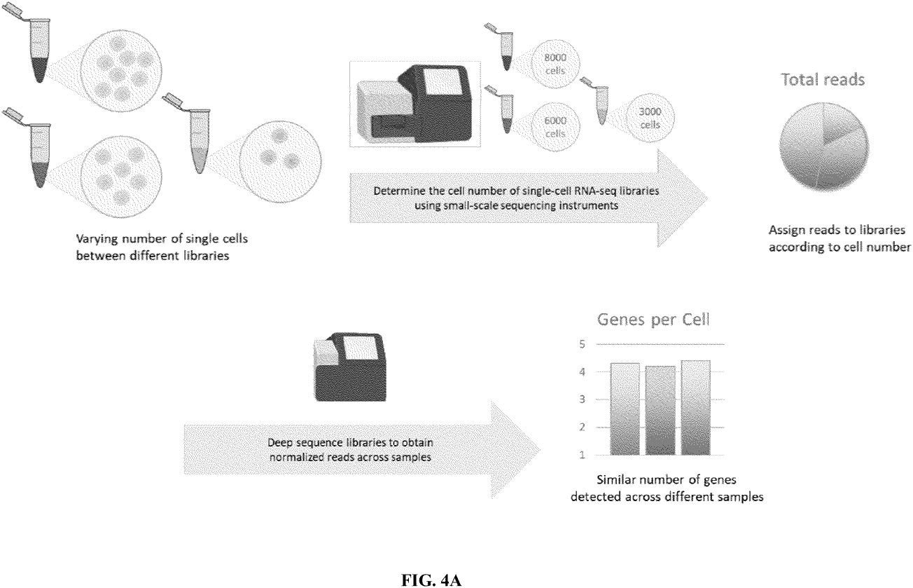

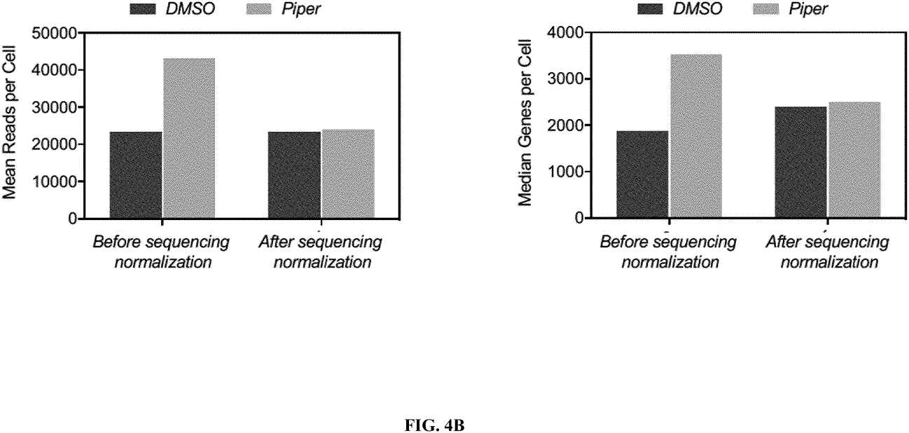

The transcriptomes of single cells treated with inhibitors or CRISPRi against same targets were isolated separately. A sequential single-cell sequencing approach (FIGS. 4A-4B, Example 5) was then applied to the samples to perform normalization of the sequence reads. A representative latent space was generated via supervised dimensionality reduction on the distinct cell populations (e.g., using UMAP or VAEs). Supervised learning (FIGS. 3A-3B) was then applied to assess drug effects by training a model on binary cell types to classify new cells by comparing classifications with original and desired states.

### Example 5 - A Sequential Single-cell Sequencing Approach for Normalizing Reads and Gene Numbers

During single cell isolation, the number of captured single cells may differ from the expected number based on counting. This can result in library read depth differences when sequencing across many samples, leading to artifacts in downstream differential expression analysis. To address this problem, a sequential single-cell sequencing approach for read normalization was developed (FIG. 4A). The number of single cells from two samples (MIAPaCa-2 cells were treated with DMSO or Piperlongumine) was first determined using small-scale sequencing instruments (MiSeq system) (FIG. 4B). After the cell number was quantified, sequence reads from a higher sequencing output sequencing instrument (NextSeq, Hiseq, or NovaSeq systems) were assigned according to the calculated cell number. Before normalization, the two single-cell samples (DMSO and Piper) resulted in varying read depths. By contrast, assigning sequencing reads based on sample cell number resulted in similar read depths across samples (FIG. 4B).

FIGS. 4A-4B show an example of a sequential single-cell sequencing approach to normalize reads and gene numbers across samples, including a schematic illustration of the normalization approach (FIG. 4A), and a number of reads and genes per cell from samples before and after the sequential single-cell sequencing approach (FIG. 4B); DMSO indicates that MIAPaCa-2 cells were treated with DMSO for 6 hours; Piper indicates that MIAPaCa-2 cells were treated with Piperlongumine for 6 hours.

### Example 6 - Machine-Learning-Driven Selection of Top Drug Candidates Based on Quantification of Single-Cell RNA-Sequencing Profiles

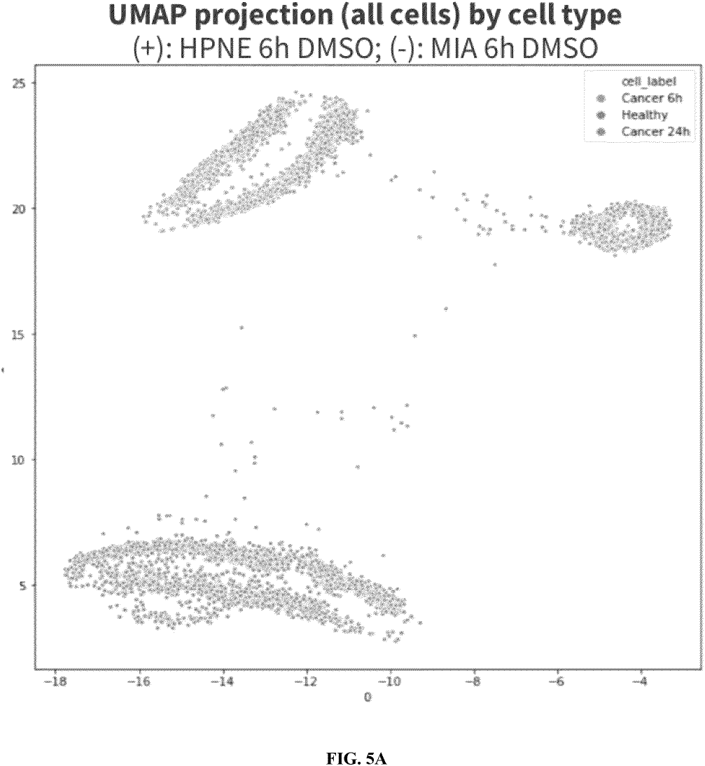

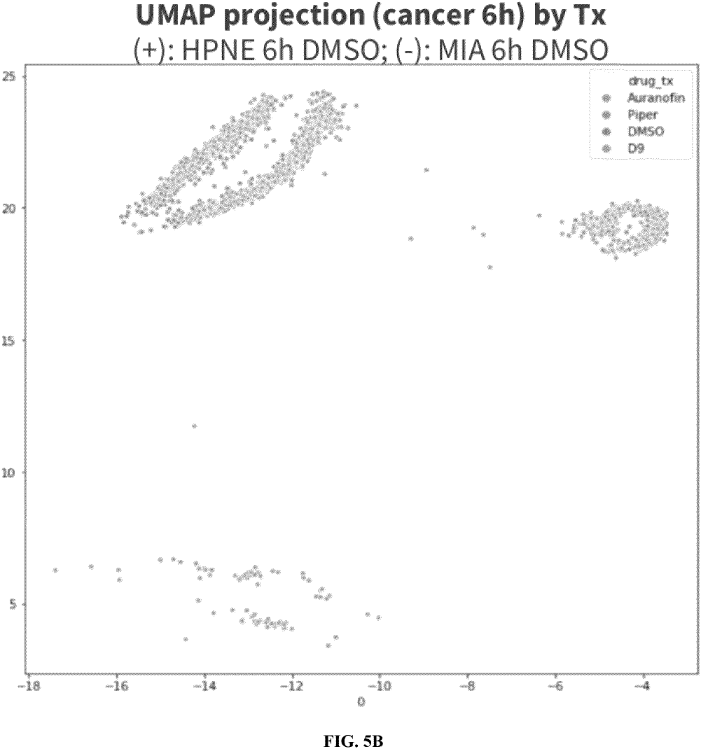

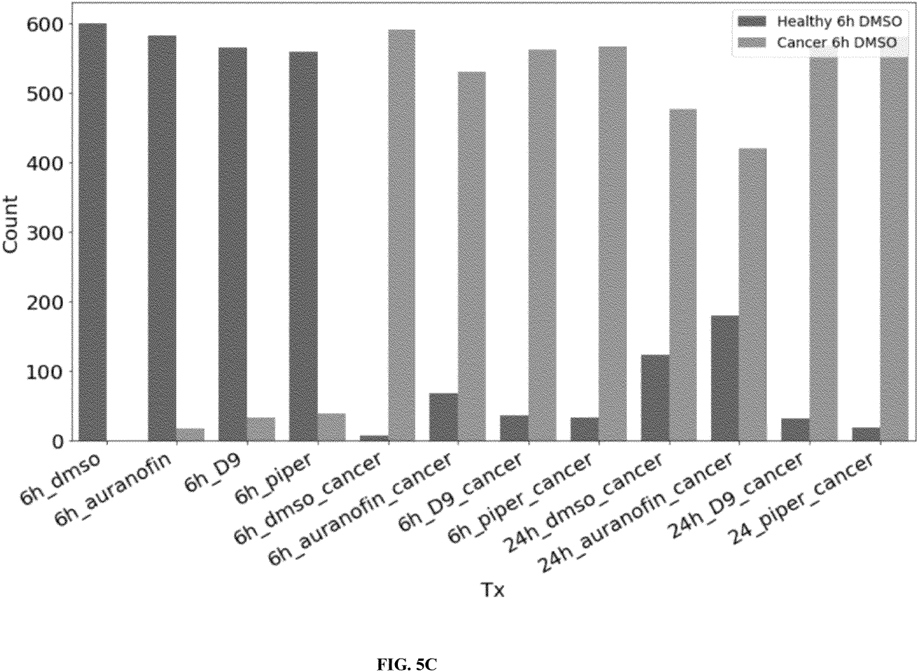

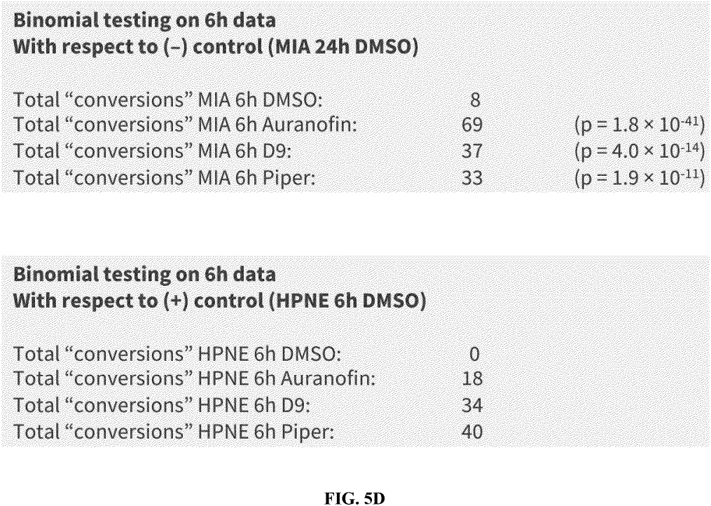

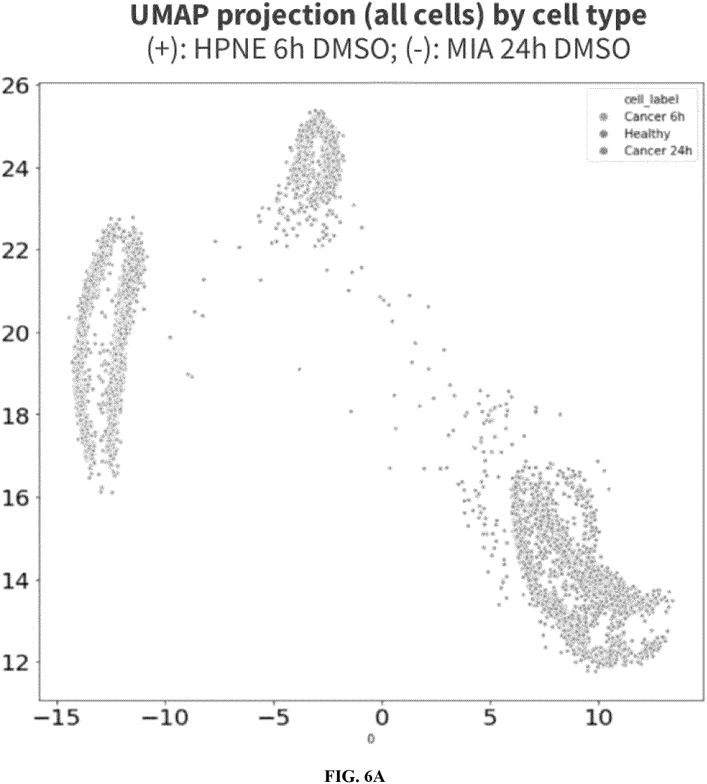

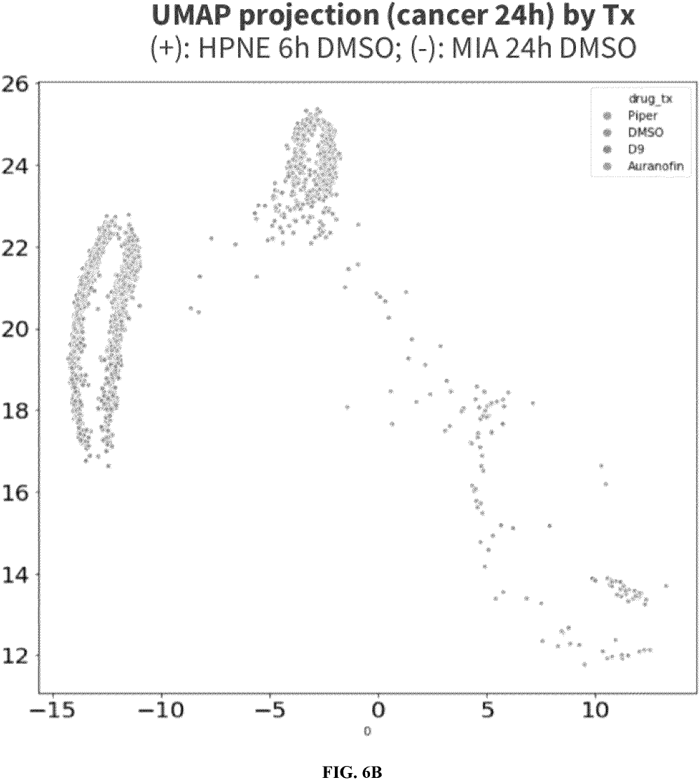

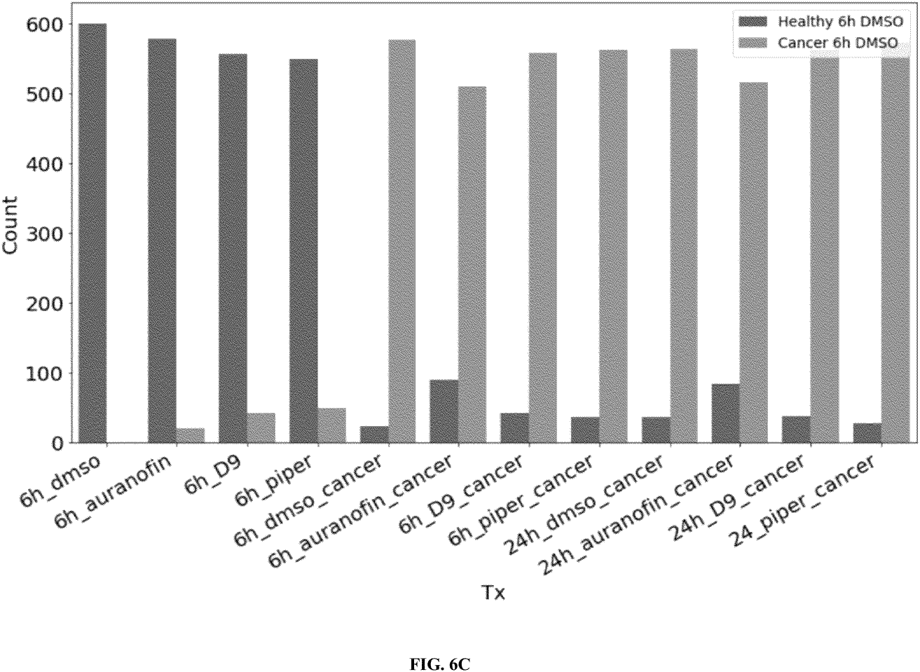

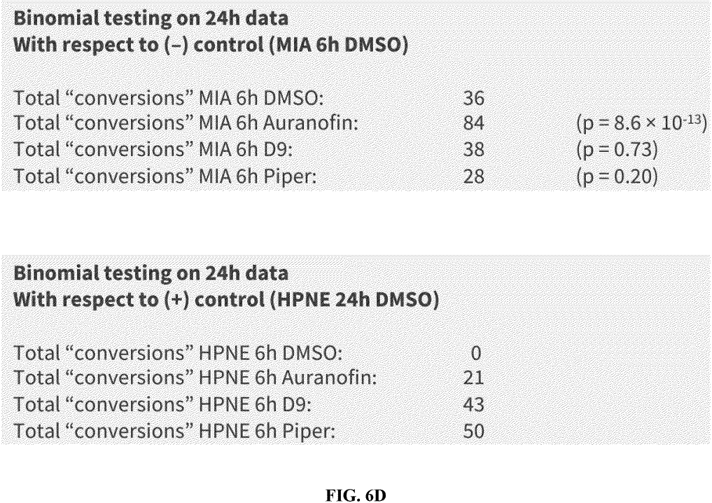

Top drug candidates are selected on the basis of their proclivity to “convert” diseased cells towards a healthy state while minimizing “conversions” of healthy cells towards a diseased state (FIGS. 5A-5D and 6A-6D). Briefly, transcriptomes of unperturbed pancreatic healthy hTERT-HPNE cells and cancer MIAPaCa-2 cells were projected onto 2-dimensional latent expression profiles via UMAP, and machine learning models were trained to discriminate between cell types in a binary fashion with an AUC > 0.98 (FIGS. 5A and 6A). MIAPaCa-2 cells were then treated for either 6 hours (FIGS. 5A-5D) or 24 hours (FIGS. 6A-6D) with drug candidates, and 2-dimensional projected transcriptomes of treated cells were subsequently classified via the trained algorithm described above. The proportion of “converted” human pancreatic cancer cells was then evaluated against a vehicle control (e.g., DMSO) via a binomial ratio test (FIGS. 5C-5D and 6C-6D). Drugs with maximal conversion of human pancreatic cancer cells and minimal conversion of healthy cells relative to a vehicle control were selected for further biological validation and development.

FIGS. 5A-5D show an example of machine-learning-driven selection of top drug candidates based on quantification of single-cell RNA-sequencing profiles (6-hour treatment). FIGS. 5A-5B show 2-dimensional UMAP projections of the human cancer pancreatic cancer cells MIAPaCa-2 and healthy pancreatic duct cells hTERT-HPNE shown by either cell type (FIG. 5A) or drug treatment (Auranofin, D9, or Piperlongumine) and duration (FIG. 5B). FIG. 5C shows machine learning classification of cells treated with either vehicle controls (DMSO) or drug candidates. Briefly, supervised machine learning algorithms were trained on 2-dimensional UMAP transcriptome profiles of pure cell types (healthy and cancer cell) to allow for binary discrimination between cell types with an AUC exceeding 0.98. Treated cells were then assigned as “cancer” or “healthy” on the basis of their resulting 2-dimensional transcriptomes following treatment. FIG. 5D shows a summary of binomial testing results for drug candidates relative to a vehicle control (DMSO).

FIGS. 6A-6D show an example of machine-learning-driven selection of top drug candidates based on quantification of single-cell RNA-sequencing profiles (24-hour treatment). FIGS. 6A-6B show 2-dimensional UMAP projections of the human cancer pancreatic cancer cells MIAPaCa-2 and healthy pancreatic duct cells hTERT-HPNE shown by either cell type (FIG. 6A) or drug treatment (Auranofin, D9, or Piperlongumine) and duration (FIG. 6B). FIG. 6C shows machine learning classification of cells treated with either vehicle controls (DMSO) or drug candidates. Briefly, supervised machine learning algorithms were trained on 2-dimensional UMAP transcriptome profiles of pure cell types (healthy and cancer cell) to allow for binary discrimination between cell types with an AUC exceeding 0.98. Treated cells were then assigned as “cancer” or “healthy” on the basis of their resulting 2-dimensional transcriptomes following treatment. FIG. 6D shows a summary of binomial testing results for drug candidates relative to a vehicle control (DMSO).

### Example 7 - Assessing On-Target Drug Effect

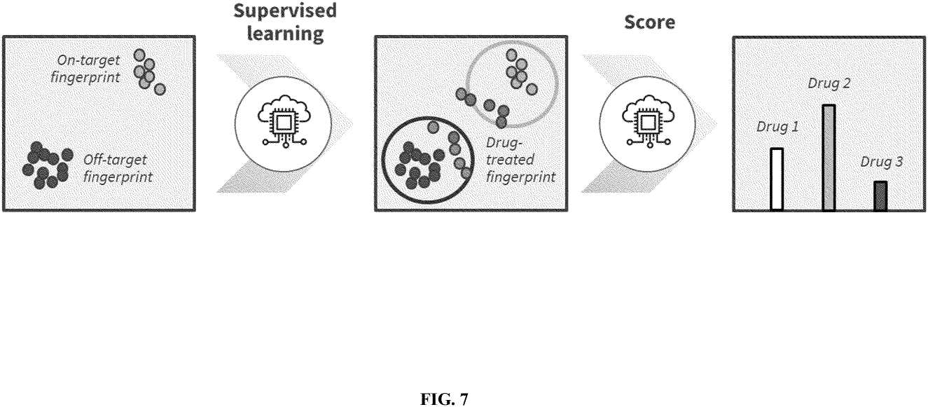

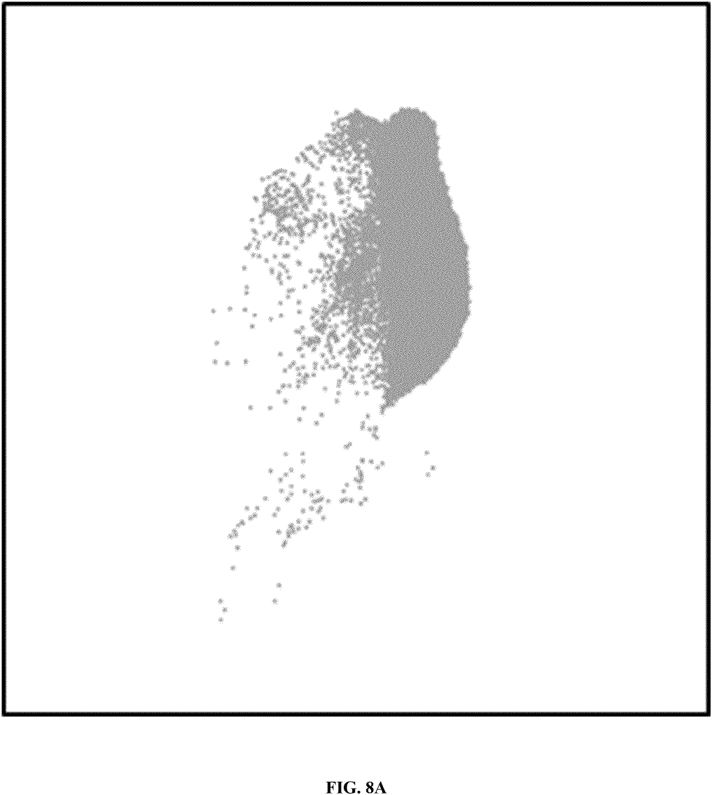

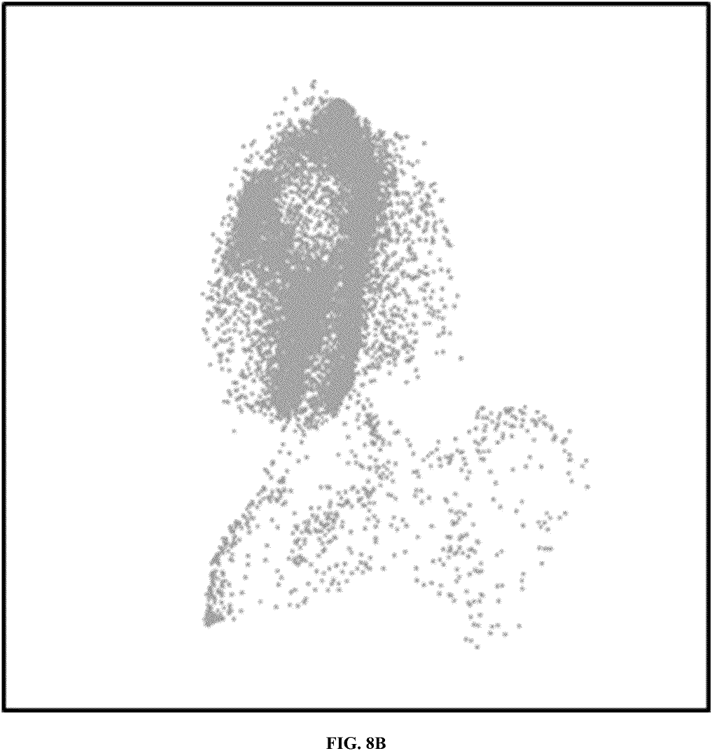

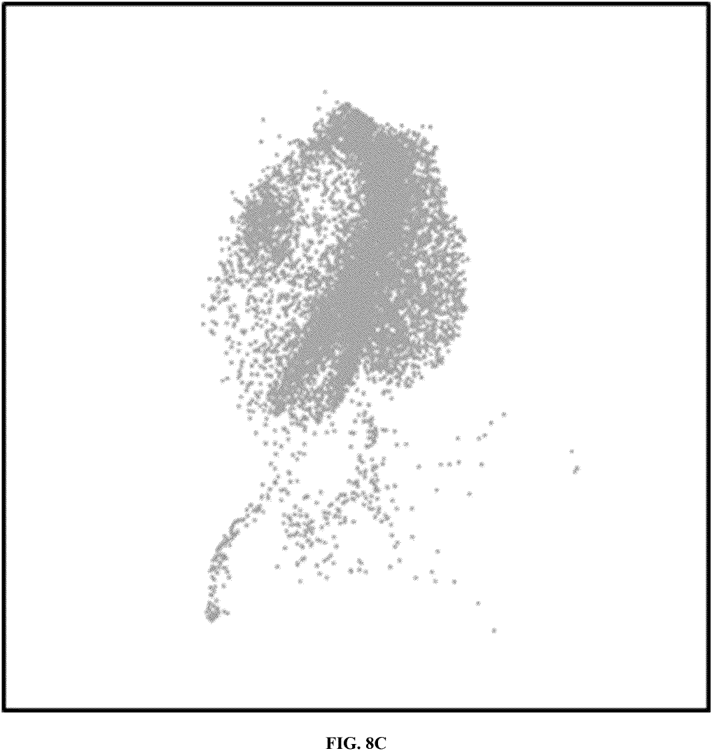

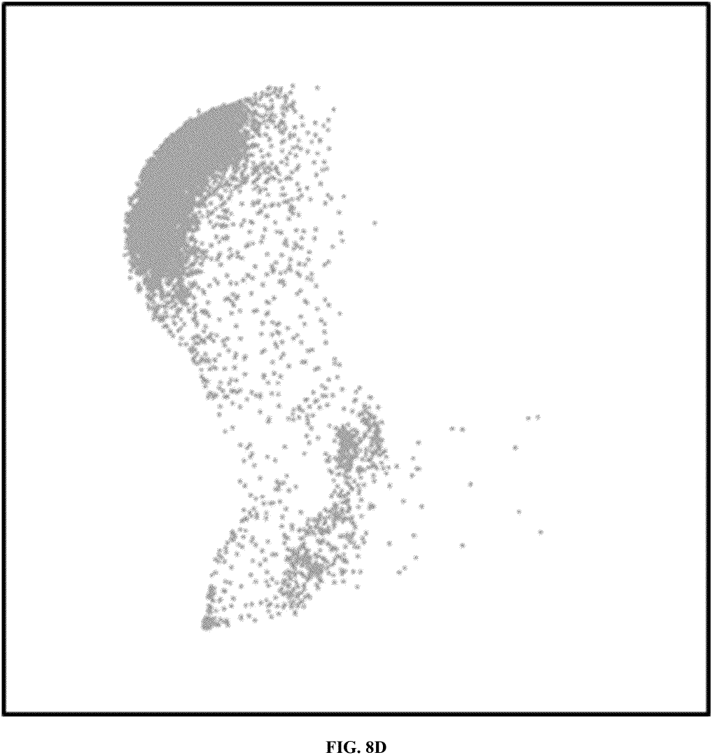

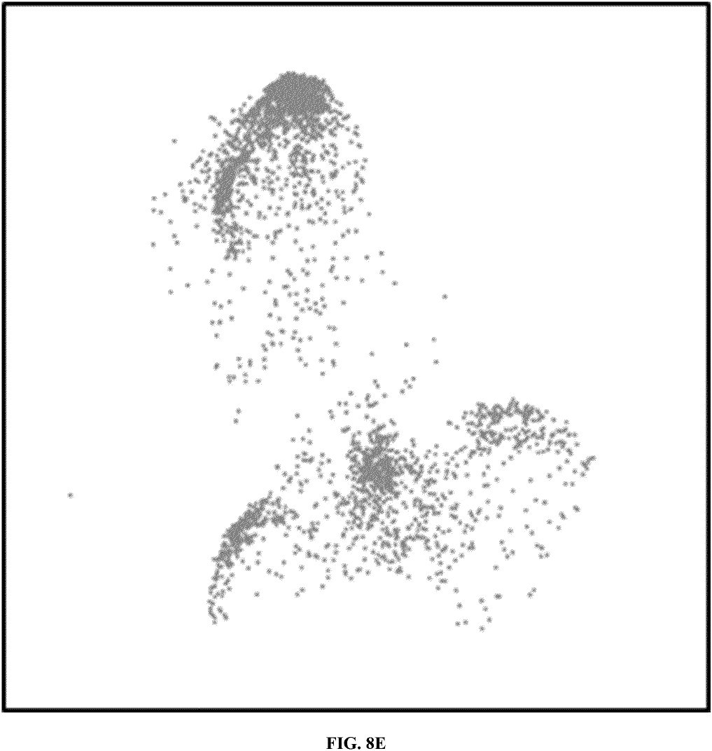

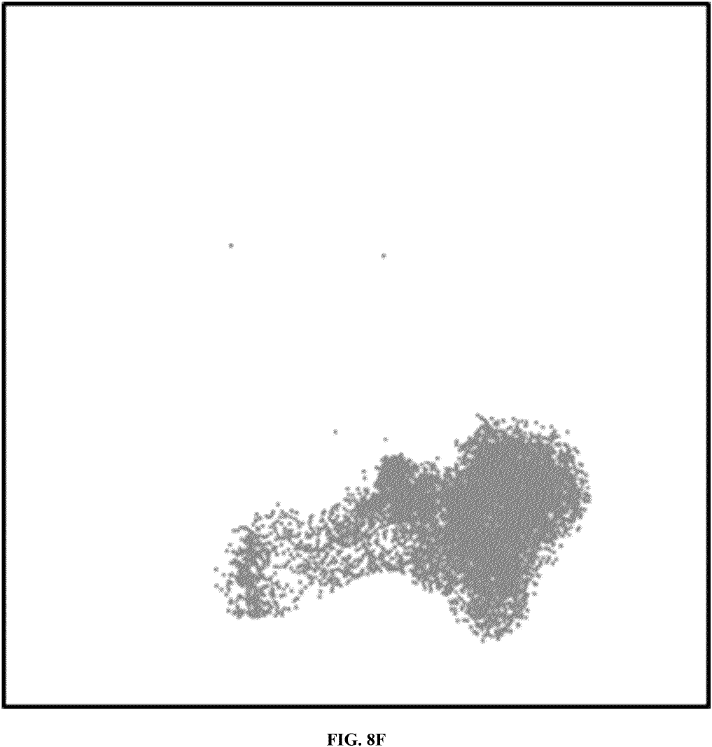

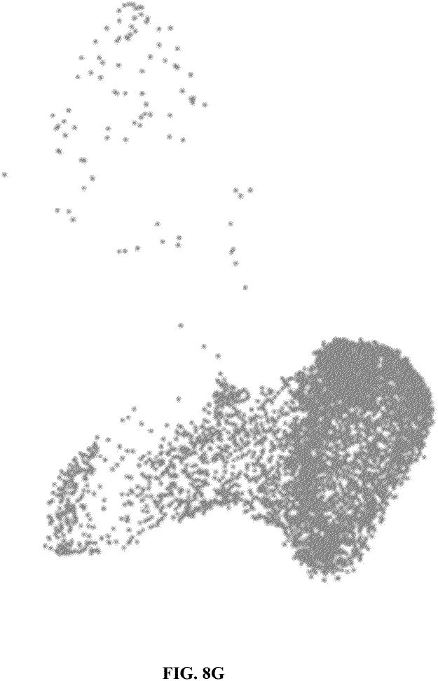

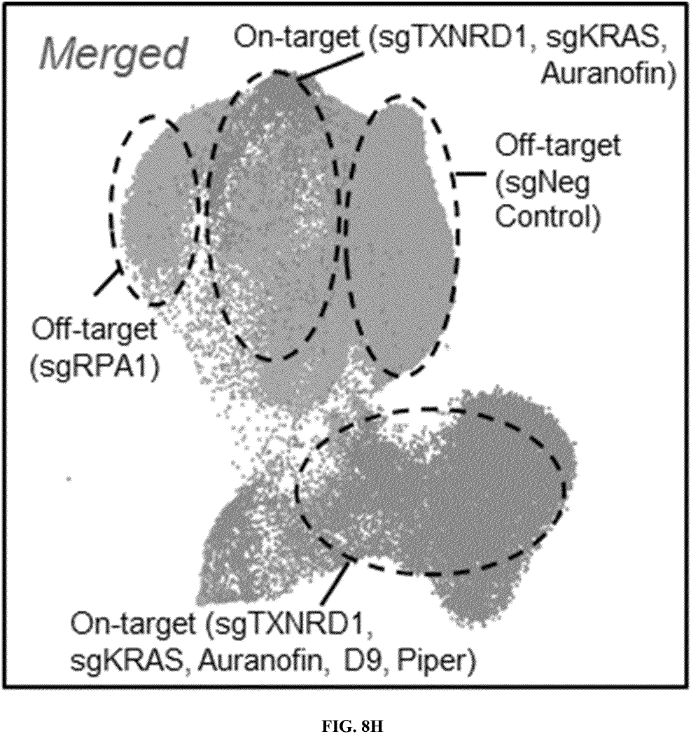

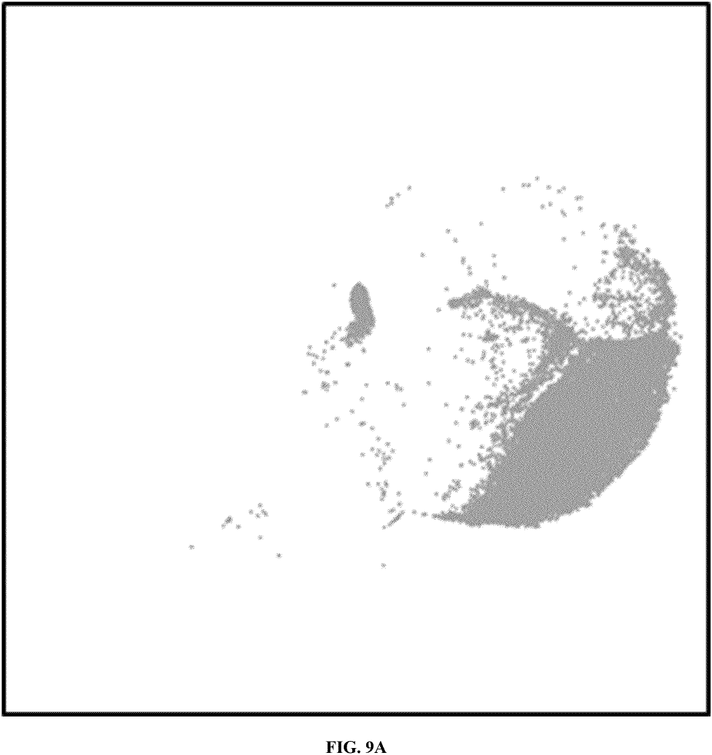

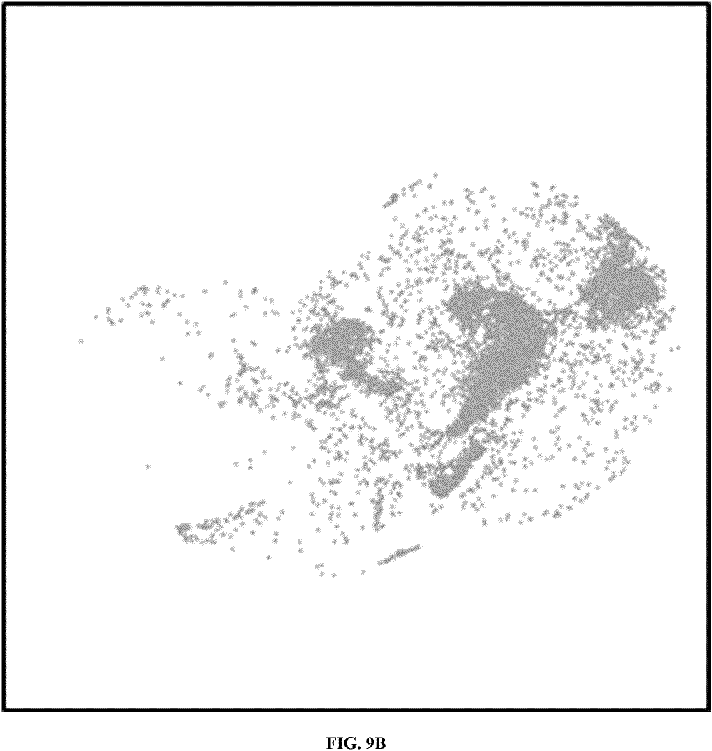

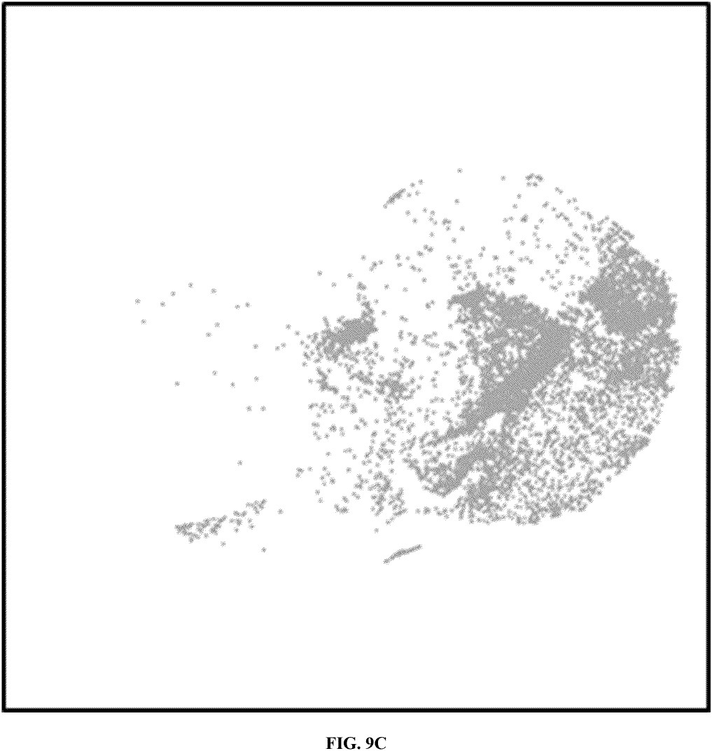

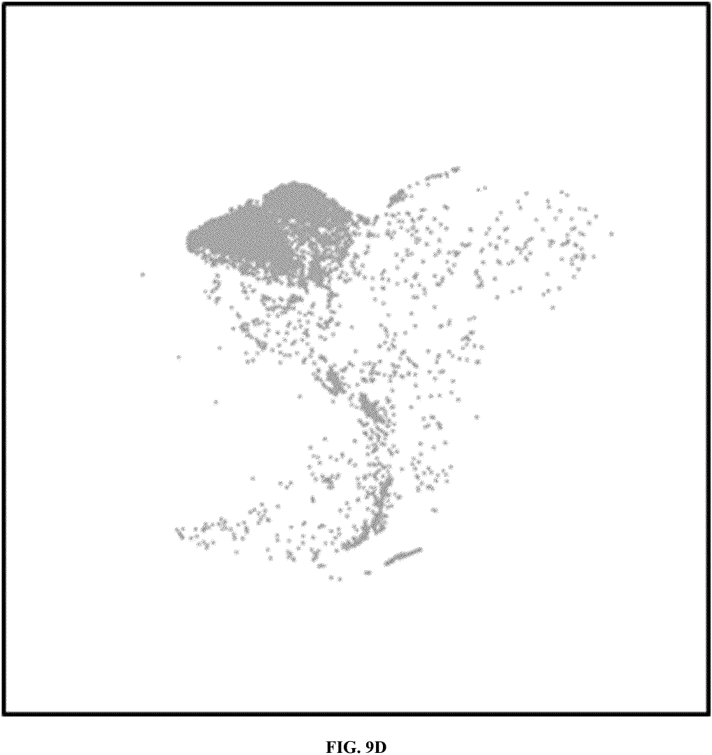

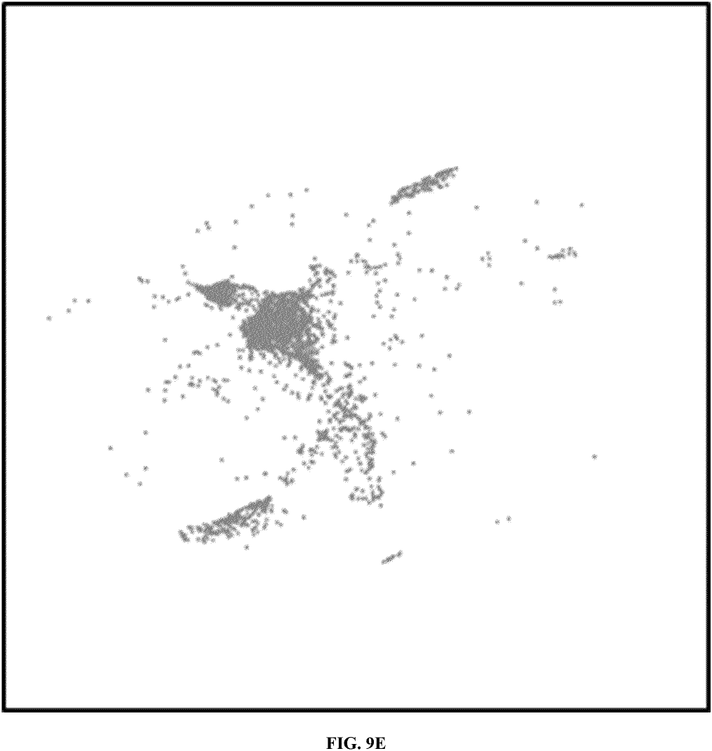

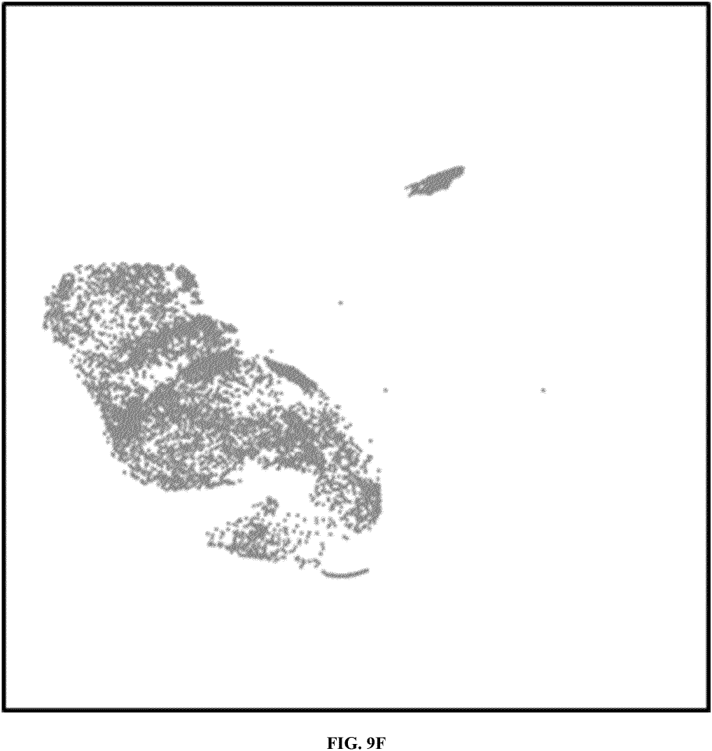

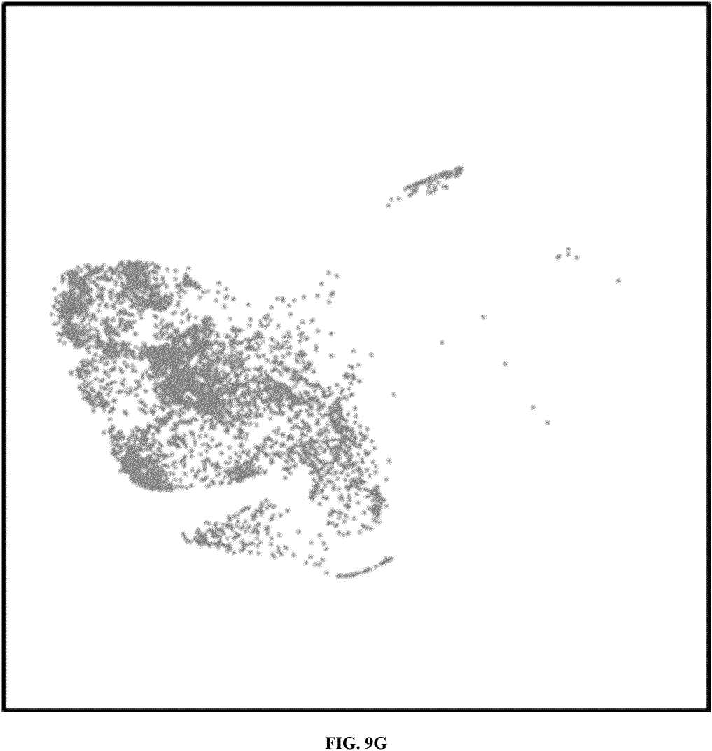

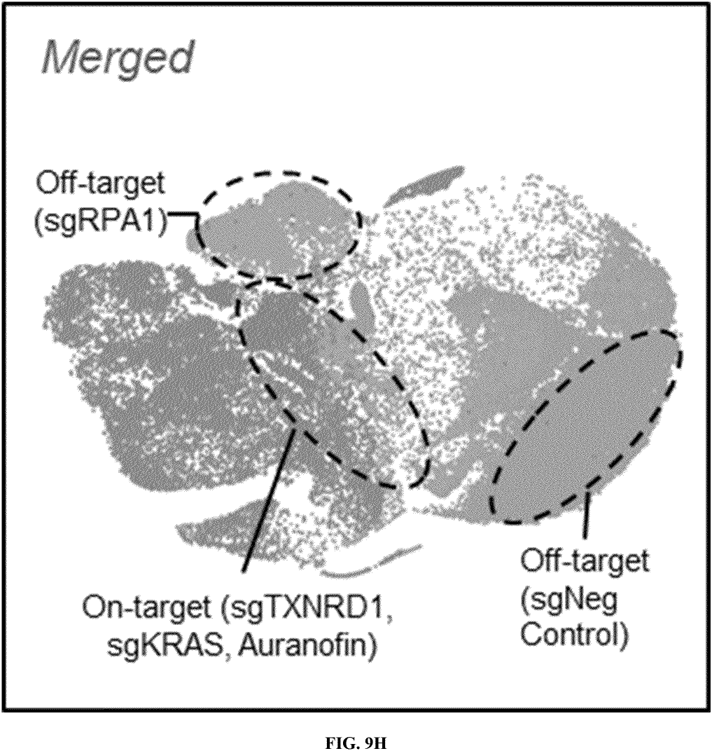

Top drug candidates were selected on the basis of their ability to match a desired fingerprint (maximal similarity of on-target fingerprint and minimal similarity of off-target fingerprint) dictated by genetic inhibition of target genes (FIG. 7). Briefly, single-cell transcriptomes of human pancreatic cancer cells MIAPaCa-2 (which may be shown to be dependent on KRAS and TXNRD1 signaling) treated with sgRNA (TXNRD1, KRAS, RPA1, negative control) or drug treatments (TXNRD1 inhibitors Auranofin, D9, or Piperlongumine) were projected to 2-dimensional latent expression profiles via UMAP (FIGS. 8A-8H) or t-SNE (FIGS. 9A-9H). Drugs with maximal similarity of sgTXNRD1 cells (and sgKRAS cells) and minimal similarity of sgRPA1 cells relative to a negative control were selected for further biological validation and development.

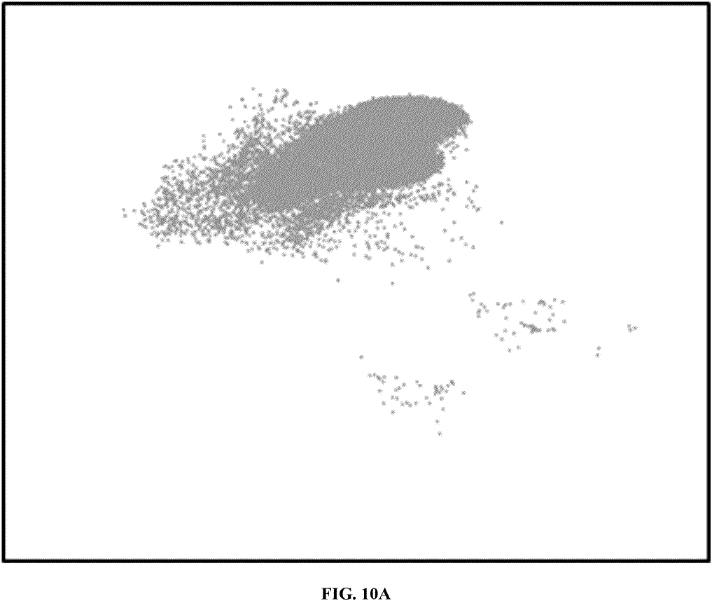

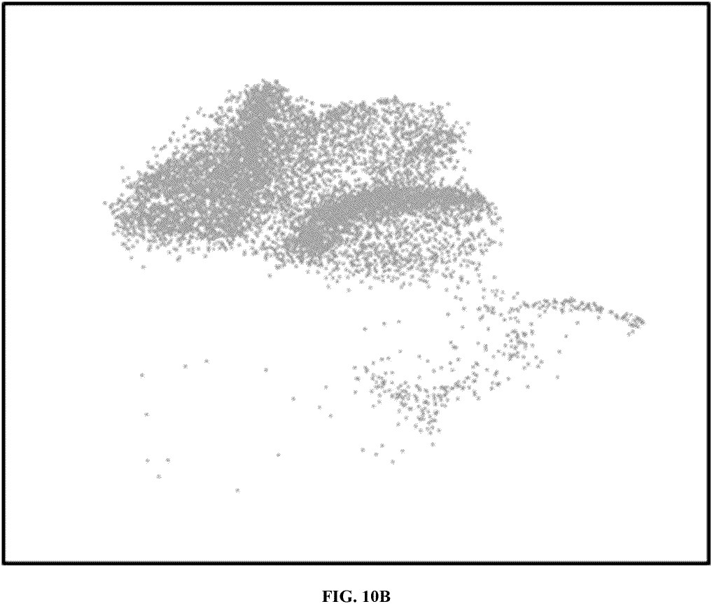

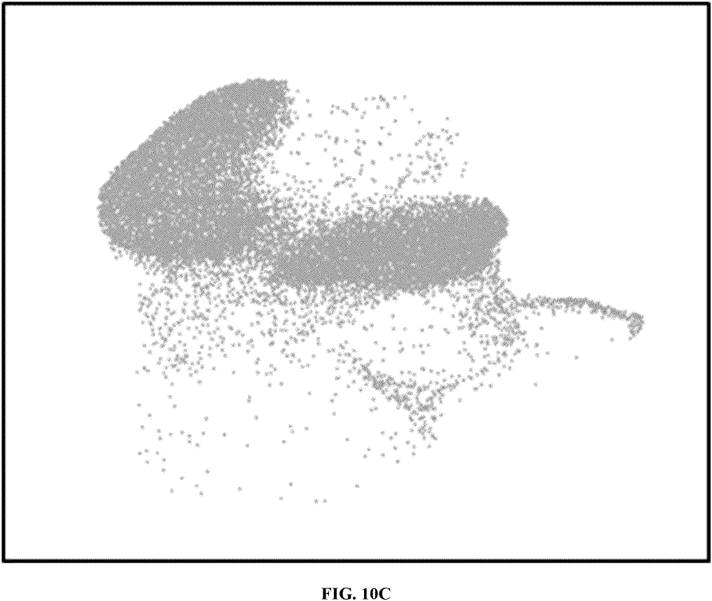

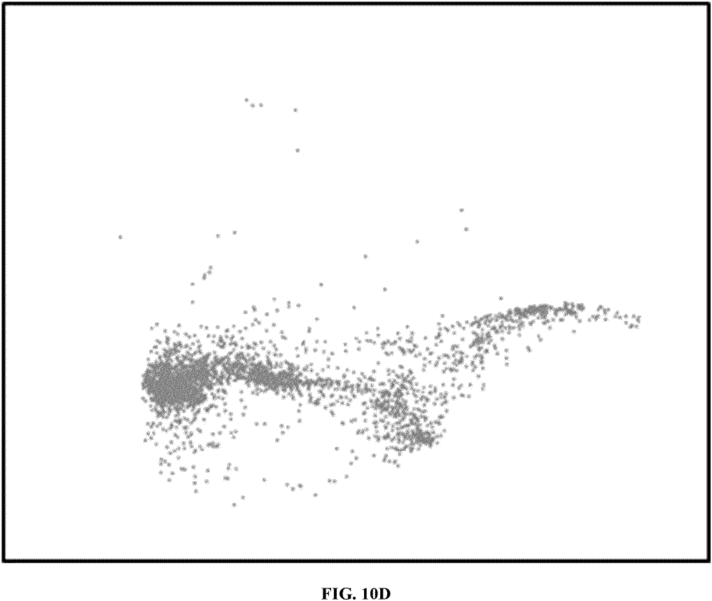

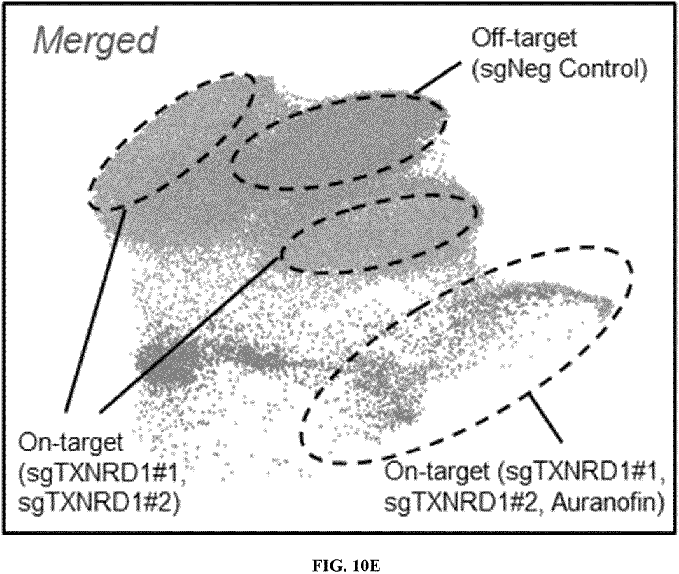

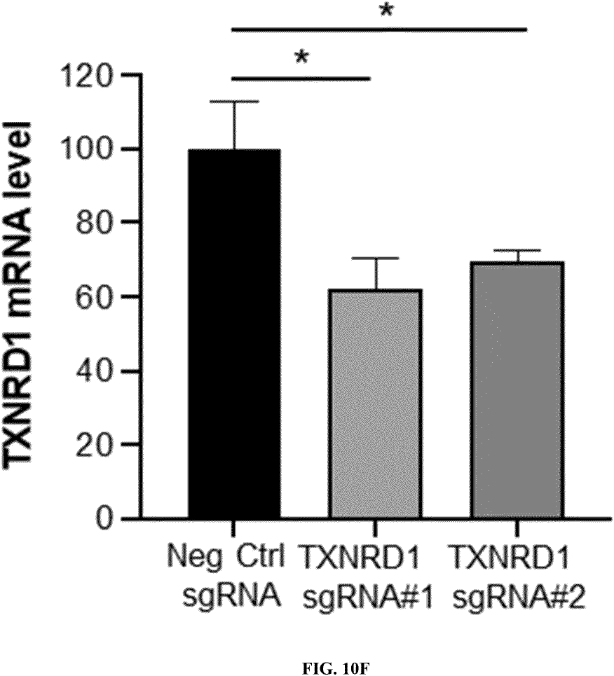

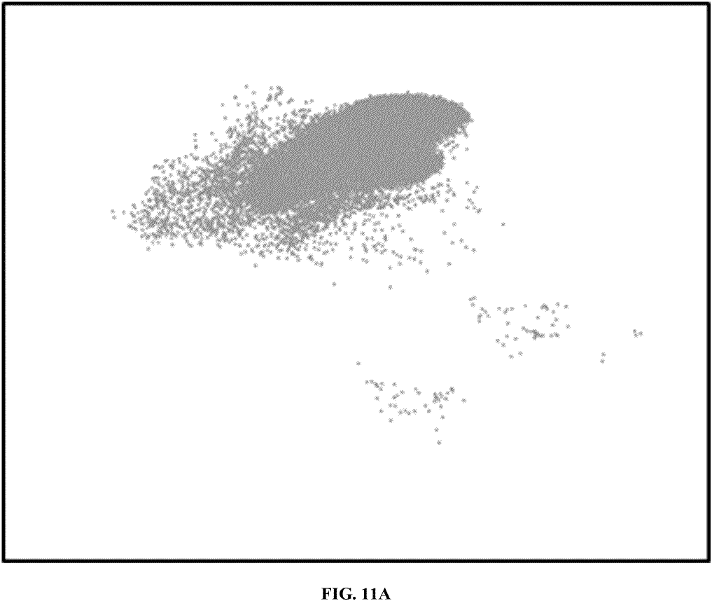

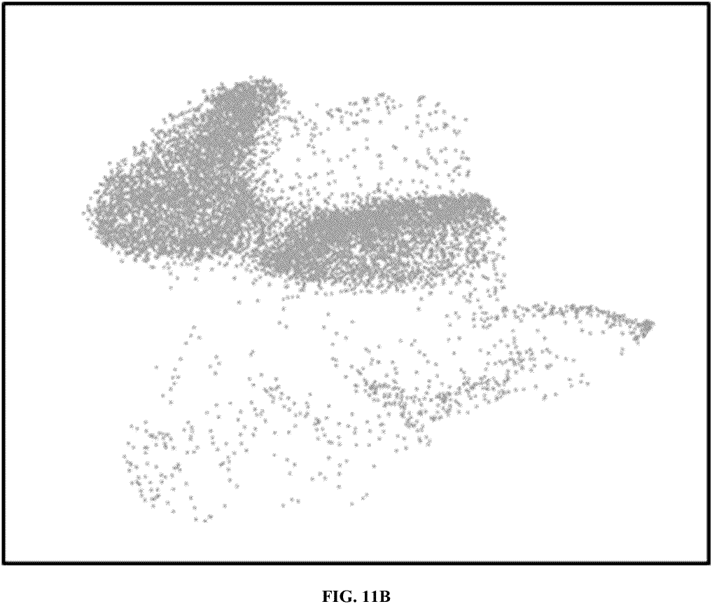

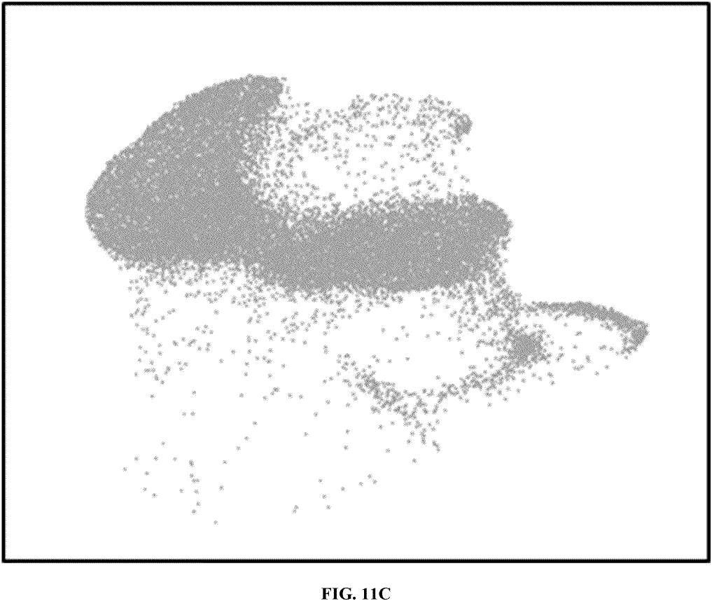

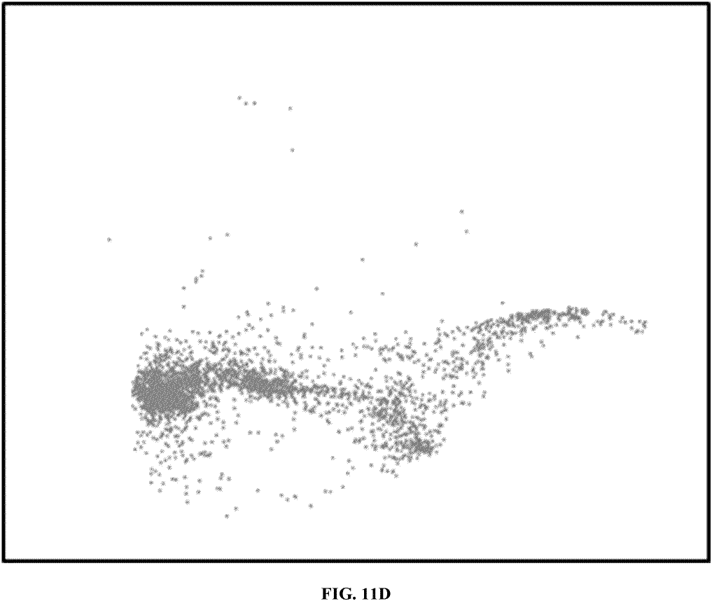

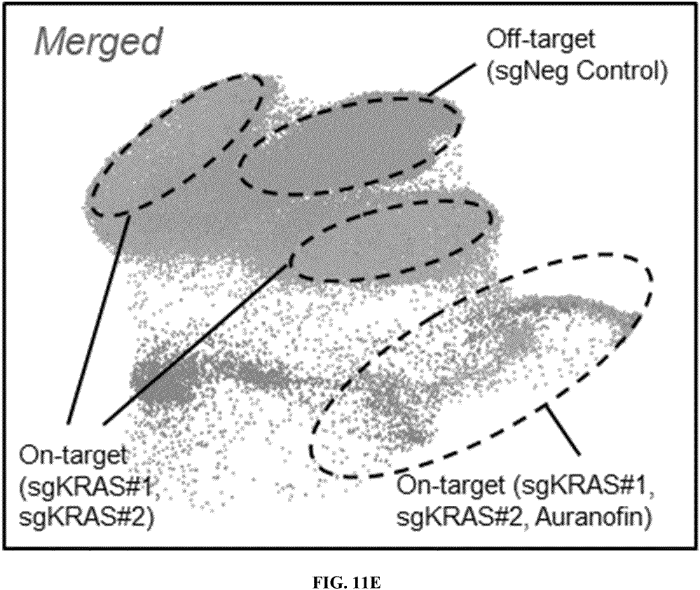

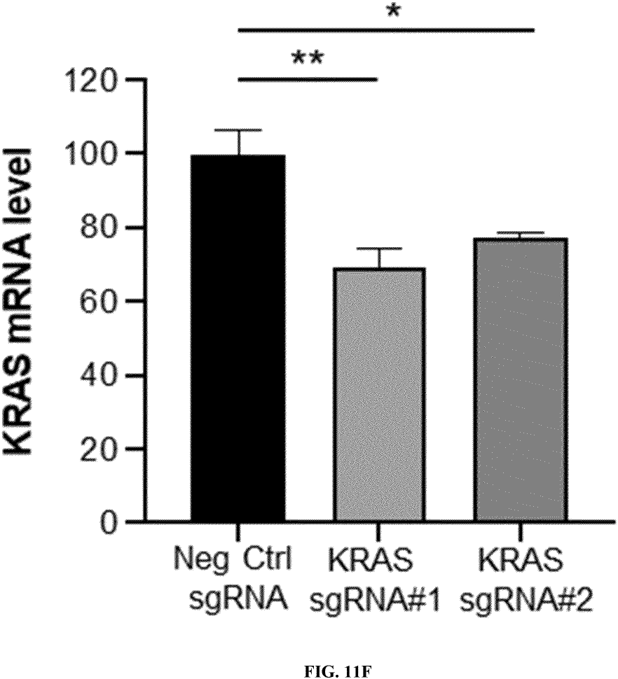

To demonstrate the reproducibility and robustness of the abovementioned method and system, we assessed drugs’ on- and off-target effects using two independent sgRNAs against the desired targets TXNRD1 (FIGS. 10A-10F) or KRAS (FIGS. 11A-11F), respectively. The two independent sgRNAs against TXNRD1 showed not only equal potency of TXNRD1 target suppression (FIG. 10F) but also highly similar single-cell transcriptomic fingerprints to assess drugs’ on- and off-target effects (FIGS. 10A-10E). Similarly, the two independent sgRNAs against KRAS showed not only equal potency of KRAS target suppression (FIG. 11F) but also highly similar single-cell transcriptomic fingerprints to assessed drugs’ on- and off-target effects (FIGS. 11A-11E).

FIG. 7 shows an illustration of supervised learning as a method for training model on binary cell types to classify new drug-treated cells by comparing classifications with cells which have on- and off-targets interrogated by CRISPR.

FIGS. 8A-8H show examples of assessing drugs’ on- and off-target effects. 2-dimensional UMAP projections of human pancreatic cancer cell lines MIAPaCa-2 (which may be shown to be dependent on KRAS and TXNRD1 signaling) were shown by sgRNA (including negative control sgRNA in FIG. 8A, KRAS sgRNA in FIG. 8B, TXNRD1 sgRNA in FIG. 8C, and RPA1 sgRNA in FIG. 8D) or drug treatments (including Auranofin in FIG. 8E, D9 in FIG. 8F, and Piperlongumine in FIG. 8G) or merged (FIG. 8H). As shown by the dash line circles in FIG. 8H, on- and off-target effects from pharmacological inhibition (TXNRD1 inhibited by Auranofin, D9, or Piperlongumine) were assessed in accordance with their ability to match an on-target fingerprint dictated by genetic inhibition (sgRNAs targeting TXNRD1 or KRAS). sgRNA targeting an essential gene RPA1 was used as a toxic control fingerprint.

FIGS. 9A-9H show examples of assessing drugs’ on- and off-target effects. 2-dimensional t-Distributed Stochastic Neighbor Embedding (t-SNE) projections of human pancreatic cancer cell lines MIAPaCa-2 (which may be shown to be dependent on KRAS and TXNRD1 signaling) were shown by sgRNA (including negative control sgRNA in FIG. 9A, KRAS sgRNA in FIG. 9B, TXNRD1 sgRNA in FIG. 9C, and RPA1 sgRNA in FIG. 9D) or drug treatments (including Auranofin in FIG. 9E, D9 in FIG. 9F, and Piperlongumine in FIG. 9G) or merged (FIG. 9H). As shown by the dash line circles in FIG. 9H, on- and off-target effects from pharmacological inhibition (TXNRD1 inhibited by Auranofin, D9, or Piperlongumine) were assessed in accordance with their ability to match an on-target fingerprint dictated by genetic inhibition (sgRNAs targeting TXNRD1 or KRAS). sgRNA targeting an essential gene RPA1 was used as a toxic control fingerprint.

FIGS. 10A-10F show the reproducibility of this method to assess drugs’ on- and off-target effects using TXNRD1 target gene as an example. 2-dimensional UMAP projections of human pancreatic cancer cell lines MIAPaCa-2 (which may be shown to be dependent on KRAS and TXNRD1 signaling) were shown by sgRNA (including negative control sgRNA in FIG. 10A, TXNRD1 #1 sgRNA in FIG. 10B, and TXNRD1 #2 sgRNA in FIG. 10C) or drug treatments (including Auranofin in FIG. 10D) or merged (FIG. 10E). As shown by the dash line circles in FIG. 10E, on- and off-target effects from pharmacological inhibition (TXNRD1 inhibited by Auranofin) were assessed in accordance with their ability to match on-target fingerprints dictated by two independent genetic inhibition (two independent sgRNAs targeting TXNRD1). Quantitative PCR (qPCR) analysis of TXNRD1 gene expression in human pancreatic cancer cell lines MIAPaCa-2 transduced with two independent sgRNAs targeting TXNRD1 is shown in FIG. 10F. Data are presented as mean ± standard deviation. Statistical significance between groups was calculated by two-tailed Student’s t-test. Significance value is P < 0.05 (*).

FIGS. 11A-11F show the reproducibility of this method to assess drugs’ on- and off-target effects using KRAS target gene as an example. 2-dimensional UMAP projections of human pancreatic cancer cell lines MIAPaCa-2 (which may be shown to be dependent on KRAS and TXNRD1 signaling) were shown by sgRNA (including negative control sgRNA in FIG. 11A, KRAS #1 sgRNA in FIG. 11B, and KRAS #2 sgRNA in FIG. 11C) or drug treatments (including Auranofin in FIG. 11D) or merged (FIG. 11E). As shown by the dash line circles in FIG. 11E, on-and off-target effects from pharmacological inhibition (Auranofin) were assessed in accordance with their ability to match on-target fingerprints dictated by two independent genetic inhibition (two independent sgRNAs targeting KRAS). Quantitative PCR (qPCR) analysis of KRAS gene expression in human pancreatic cancer cell lines MIAPaCa-2 transduced with two independent sgRNAs targeting KRAS is shown in FIG. 11F. Data are presented as mean ± standard deviation. Statistical significance between groups was calculated by two-tailed Student’s t-test. Significance values are P < 0.05 (*) and P < 0.01 (**).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1-43. (canceled)

44. A method for determining an effectiveness of a drug, comprising:
(a) generating a latent space representation of nucleic acid sequence data for a plurality of diseased cells and a plurality of normal cells of a cell type, wherein said latent space represents a plurality of phenotypic states of said cell type;
(b) identifying, based at least in part on a topology of said latent space, a target genomic region of said cell type;
(c) mapping sequence data of a first cell of said cell type to said latent space to yield a first latent space representation, wherein said target genomic region of said first cell has been modified, and wherein said first cell exhibited a first phenotypic state prior to said modification;
(d) mapping sequence data of a second cell of said cell type to said latent space to yield a second latent space representation, wherein said second cell has been exposed to said drug, and wherein prior to said second cell being exposed to said drug, said second cell exhibited said first phenotypic state; and
(e) determining, based at least in part on said first latent space representation and said second latent space representation, said effectiveness of said drug.

45. The method of claim 44, wherein (a) further comprises using a supervised dimensionality reduction algorithm to generate said latent space representation.

46. The method of claim 45, wherein said supervised dimensionality reduction algorithm comprises a uniform manifold approximation and projection (UMAP) algorithm, a t-distributed stochastic neighbor embedding (t-SNE) algorithm, or a variable autoencoder.

47. The method of claim 1, wherein said first phenotypic state comprises a cancerous state.

48. The method of claim 1, wherein said first phenotypic state comprises an intermediate state, wherein said intermediate state is a fibroblast state or a progenitor cell state.

49. The method of claim 1, wherein (e) further comprises measuring (i) a shift in said latent space representation of said first cell from said modification, and (ii) a shift in said latent space representation of said second cell from said exposure to said drug; and mathematically relating (i) to (ii).

50. The method of claim 49, wherein said measuring further comprises using a supervised learning algorithm, wherein said supervised learning algorithm is a support vector machine, a random forest, logistic regression, a Bayesian classifier, or a convolutional neural network.

51. The method of claim 1, further comprising:
mapping nucleic acid sequence data of a plurality of additional cells of said cell type to said latent space, wherein each cell of said plurality of additional cells has been exposed to a respective drug of a plurality of drugs;
determining, based at least in part on said latent space representation of said first cell and latent space representations of said plurality of additional cells, an effectiveness of each drug; and
electronically outputting a ranking of said plurality of drugs based at least in part on said effectiveness of each drug.

52. The method of claim 1, wherein said drug is selected from the group consisting of: a compound, an inhibitor, and an antibody.

53. The method of claim 1, wherein at least one of said sequence data of said first cell of said cell type and said sequence data of said second cell of said cell type is generated by single-cell sequencing.

54. The method of claim 1, wherein said modification in (c) further comprises use of a genetic editing unit, wherein said genetic editing is performed with a genetic editing unit selected from the group consisting of a CRISPR system, a CRISPRi system, a CRISPRa system, a RNAi system, and a shRNA system.

55. The method of claim 1, wherein said modification in (c) further comprises use of a single-guide RNA (sgRNA) that targets at least a portion of said target genomic region.

56. The method of claim 1, wherein (e) further comprises comparing said first latent space representation to said second latent space representation.

57. The method of claim 56, wherein (e) further comprises determining said effectiveness of said drug based at least in part on determining a maximal similarity of said first latent space representation to an on-target latent space representation or a minimal similarity of said first latent space representation to an off-target latent space representation.

58. A method for determining an effectiveness of a drug, comprising:
(a) generating a latent space representation of nucleic acid sequence data for a plurality of diseased cells and a plurality of normal cells of a cell type, wherein said latent space represents a plurality of phenotypic states of said cell type;
(b) identifying, based at least in part on a topology of said latent space, a genomic region that facilitates reprogramming of said cell type from a first phenotypic state to a second phenotypic state of said plurality of phenotypic states;
(c) mapping sequence data of a first cell of said cell type to said latent space to yield a first latent space representation, wherein said first cell has been reprogrammed from said first phenotypic state to said second phenotypic state;
(d) mapping sequence data of a second cell of said cell type to said latent space to yield a second latent space representation, wherein said second cell has been exposed to said drug, and wherein prior to said second cell being exposed to said drug, said second cell exhibited said first phenotypic state; and
(e) determining, based at least in part on said first latent space representation and said second latent space representation, said effectiveness of said drug.

59. The method of claim 58, wherein (a) further comprises using a supervised dimensionality reduction algorithm to generate said latent space representation.

60. The method of claim 59, wherein said supervised dimensionality reduction algorithm comprises a uniform manifold approximation and projection (UMAP) algorithm, a t-distributed stochastic neighbor embedding (t-SNE) algorithm, or a variable autoencoder.

61. The method of claim 58, wherein (b) further comprises performing non-linear cell trajectory reconstruction on said latent space to construct an inferred maximum likelihood progression trajectory between said first phenotypic state and said second phenotypic state.

62. The method of claim 61, wherein performing said non-linear cell trajectory reconstruction further comprises applying a reverse graph embedding algorithm to said latent space.

63. The method of claim 58, wherein said first phenotypic state comprises a cancerous state, and wherein said second phenotypic state comprises a wild-type state.

64. The method of claim 58, wherein said second phenotypic state is an intermediate state, wherein said intermediate state is a fibroblast state or a progenitor cell state.

65. The method of claim 58, wherein said first cell has been reprogrammed from said first phenotypic state to said second phenotypic state using a genetic editing unit, wherein said genetic editing unit is selected from the group consisting of a CRISPR system, a CRISPRi system, a CRISPRa system, a RNAi system, and a shRNA system.

66. The method of claim 58, wherein (e) further comprises measuring (i) a shift in said latent space representation of said first cell from said editing and (ii) a shift in said latent space representation of said second cell from said exposure to said drug; and mathematically relating (i) to (ii).

67. The method of claim 66, wherein said measuring further comprises using a supervised learning algorithm, wherein said supervised learning algorithm is a support vector machine, a random forest, logistic regression, a Bayesian classifier, or a convolutional neural network.

68. The method of claim 58, further comprising:
mapping nucleic acid sequence data of a plurality of additional cells of said cell type to said latent space, wherein each cell of said plurality of additional cells has been exposed to a respective drug of a plurality of drugs;
determining, based at least in part on said latent space representation of said first cell and latent space representations of said plurality of additional cells, an effectiveness of each drug; and
electronically outputting a ranking of said plurality of drugs based at least in part on said effectiveness of each drug.

69. The method of claim 58, wherein said drug is selected from the group consisting of: a compound, an inhibitor, and an antibody.

70. The method of claim 58, wherein at least one of said sequence data of said first cell of said cell type and said sequence data of said second cell of said cell type is generated by single-cell sequencing.

71. A system for determining an effectiveness of a drug, comprising:
a database that comprises nucleic acid sequence data for a plurality of diseased cells and a plurality of normal cells of a cell type; and
one or more computer processors that are individually or collectively programmed to:
(i) generate a latent space representation of said nucleic acid sequence data, wherein said latent space represents a plurality of phenotypic states of said cell type;
(ii) identify, based at least in part on a topology of said latent space, a genomic region that facilitates reprogramming of said cell type from a first phenotypic state to a second phenotypic state of said plurality of phenotypic states;
(iii) map sequence data of a first cell of said cell type to said latent space to yield a first latent space representation, wherein said first cell has been reprogrammed from said first phenotypic state to said second phenotypic state;
(iv) map sequence data of a second cell of said cell type to said latent space to yield a second latent space representation, wherein said second cell has been exposed to said drug, and wherein prior to said second cell being exposed to said drug, said second cell exhibited said first phenotypic state; and
(v) determine, based at least in part on said first latent space representation and said second latent space representation, said effectiveness of said drug.

72. A non-transitory computer-readable medium comprising machine-executable code that, upon execution by one or more computer processors, implements a method for determining an effectiveness of a drug, said method comprising:
(a) generating a latent space representation of nucleic acid sequence data for a plurality of diseased cells and a plurality of normal cells of a cell type, wherein said latent space represents a plurality of phenotypic states of said cell type;
(b) identifying, based at least in part on a topology of said latent space, a genomic region that facilitates reprogramming of said cell type from a first phenotypic state to a second phenotypic state of said plurality of phenotypic states;
(c) mapping sequence data of a first cell of said cell type to said latent space to yield a first latent space representation, wherein said first cell has been reprogrammed from said first phenotypic state to said second phenotypic state;
(d) mapping sequence data of a second cell of said cell type to said latent space to yield a second latent space representation, wherein said second cell has been exposed to said drug, and wherein prior to said second cell being exposed to said drug, said second cell exhibited said first phenotypic state; and
(e) determining, based at least in part on said first latent space representation and said second latent space representation, said effectiveness of said drug.
